(19) United States
(12) Patent Application Publication
(10) Pub. No.: US 2024/0069020 A1
(43) Pub. Date: Feb. 29, 2024

(54) FERROFLUID-BASED ASSAY METHODS, AND SYSTEMS FOR PARASITE EGGS OR OOCYSTS DETECTION

(71) Applicant: Ancera Inc., Branford, CT (US)

(72) Inventors: Mary K.H. SMITH, Clinton, CT (US); Arjun GANESAN, Newington, CT (US); John C. VOYTA, Berlin, MA (US)

(73) Assignee: Ancera Inc., Branford, CT (US)

(21) Appl. No.: 18/275,594

(22) PCT Filed: Feb. 2, 2022
(86) PCT No.: PCT/US2022/014987

§ 371 Date: Aug. 2, 2023

Related U.S. Application Data
(60) Provisional application No. 63/144,936, filed on Feb. 2, 2021.

(51) Int. Cl. G01N 33/569; B01L 3/00; G01N 33/543

(52) U.S. Cl. CPC G01N 33/56905; B01L 3/502761; G01N 33/54386; B01L 2200/0647; B01L 2200/16; G01N 2333/45; G01N 2333/455

(57) ABSTRACT

Embodiments of the present disclosure are directed to ferrofluid-based assay methods, systems and device for detecting one or more parasite oocyst or egg, and more specifically, to detecting parasite oocysts or eggs in fecal matter of livestock, so as to determine parasitic infections within such livestock. Such embodiments are configured to help in the ability to maintain healthy livestock for human consumption.

### RELATED APPLICATIONS

The present application claims priority to, and the benefit of, U.S. Provisional Patent Application No. 63/144,936, filed on Feb. 2, 2021, the content of which is incorporated by reference herein in its entirety.

The current disclosure is related to U.S. Pat. Nos. 8,961,878, and 9,999,855, and PCT publication nos. WO2014144340, WO2014144782, WO2014144810, WO2014145765, WO2014165317, WO2016210348. WO2017004595, WO2018026605, WO2019117877, and WO2022/015845. Each of the foregoing disclosures incorporated by reference herein in its entirety.

### SUMMARY OF THE DISCLOSURE

Embodiments of the present disclosure are directed to assay methods, systems, and devices for detecting parasites and their ova in varied substrates such as environmental samples, produce rinses, tissue samples, and fecal material. Such embodiments are configured to help in the ability to maintain the health and productivity of humans and animals through better detection of organisms. Examples of parasitic ova that are detectable by this methodology include:
Cryptosporidium parvum in feces from mammals (e.g. cattle and humans), environmental water samples, or food production (e.g. produce wash water or commercial seafood harvests);
Toxoplasma gondii, another zoonotic parasite found in human and animal tissues, fecal samples, and environmental samples; and
coccidian parasites (e.g. Eimeria spp., Isosopora spp.)

Accordingly, in some embodiments, methods, systems, and devices are configured to determine whether or not, and to what extent, Eimeria oocysts are contained in livestock, and in particular, in poultry feces. So that samples can be adequately analyzed by one and/or another of assay systems according to any one or more of the Prior Embodiments (as well as any disclosed in the '712 application), prior to loading a sample(s) into an assay system, or a component thereof (e.g., an assay cartridge), the fecal sample is (in some embodiments) homogenized into a slurry. Accordingly, for some embodiments, a ratio of Ig of fecal material mixed with 10 mL of solution permits the sample to flow through the cartridge. In some embodiments, water, sugar solution, saline, salt solutions, aqueous bases (e.g NaOH), acids (e.g. sulfuric acid, concentrated sulfuric acid, etc.) and/or sodium hypochlorite, as well as various organic solvents, could be used (in various proportions).

A common alternative to sampling clean poultry feces samples for testing is to gather other samples that have feces in them, such as litter, boot-swabs, or boot-socks. These types of samples are commonly used in the poultry industry to assess the average pathogen levels in a flock of poultry. These samples can be processed and tested for parasite, or parasite oocyst or egg levels by methods as described in this application.

Accordingly, in some embodiments, an integrated, automated diagnostic assay system is provided which is configured to at least one of detect and enumerate one or more parasite oocysts or eggs from a plurality of samples. Such a system includes corresponding methodology/processes/steps/functionality (at least some of which is referenced below).

Such embodiments (as well as other embodiments) may further include one and/or another, and in some embodiments, a plurality of, and in some embodiments, a majority of, and in some embodiments, substantially all of, and in still further embodiments, all of (if not mutually exclusive), of the following steps, features, functionality, structure, and/or clarification, yielding yet further embodiments:
the system comprises a fluidic assay system;
the system further comprises at least one of: imaging means; image processing means; and assay processing means;
the assay processing means (see immediately above) comprises at least one of reagents, and controls,
the system is configured to separate the cells;
the system is configured to move or otherwise locate oocysts (and in some embodiments, cells) to an imaging window, such that, and according to some embodiments, the oocysts (and/or cells) are pushed (via, e.g., action of ferrofluid and associated magnetic fields) to a surface for imaging thereof;
any parasite oocysts or eggs contained within at least one of the plurality of samples are at least one of: labeled and visualized by fluorescence, or unlabeled and visualized by respective intrinsic auto-fluorescence;
the assay system is a ferrofluidic based assay system, such that:
   the samples are mixed with ferrofluid prior to being flowed past a capture region of the system; or, the sample mixed with ferrofluid can be flowed (pumped) into an imaging window without a capture event;
   the system establishes electromagnetic fields to act on the ferrofluid so as to direct at least one of parasites oocysts or eggs contained in the sample to a specific area or location; and/or
   the ferrofluid may also include at least one density modifying agent, where the density modifying agent can comprise at least one of a concentrated sugar solution, concentrated salt solution, or Sheather's solution;
each sample can be at least one of processed and mixed with sodium hydroxide, where the sodium hydroxide can be mixed with each sample by mixing equal volumes of sample and sodium hydroxide solution, optionally incubating the mixture prior to flowing the mixture over a capture region of the system, and the concentration of sodium hydroxide can be: between 0.001 molar (M) and 2M, between 0.2M and 1.2M, or at about 1M (one of skill in the art will appreciate that in place of NaOH, other strong bases or detergents (anionic, non-ionic, or cationic) can be used, with a concentration of between 0.005% and 0.5%);
incubation time (see immediately above) can be between less than 45 minutes, between 1-45 minutes, or at about 30 minutes, prior to the samples being analyzed;
fecal samples can be added to ferrofluid at a predetermined ratio, ranging from 1/20 to 2/1 ratio of ferrofluid to prepared sample;
parasites or oocysts contained within at least one of the plurality of samples may be visualized using a hemocytometer and/or a McMaster chamber,
   at least one of the hemocytometer and McMaster chamber can be configured to cause fluorescence excitation/emission of fluorophore labeled parasites and/or oocysts in the samples using wavelengths specific to the fluorophore used; and/or
   fluorophore labeled parasites and/or oocysts can be visualized in at least one of the hemocytometer and McMaster chamber by fluorescence excitation/emission of the fluorophore using wavelengths specific to the fluorophore used
any capture region of the system can be coated with a binding agent configured to bind with surface features on at least one of the parasites and oocysts;
   the binding agent can comprise at least one of an antibody, aptamer, lectin, and mucin;
   the binding agent can be bound to the capture region via at least one of avidin, streptavidin, neutravidin, and any other modified binding agent;
a cartridge can be used that includes a plurality of channels/lanes each with at least one capture region/zone, and a window proximate each capture region/zone;
a cartridge that can be used that includes a plurality of channels/lanes and a window proximate each channel/lane;
the system is configured to flow at least one of the plurality of samples into a cartridge for a predetermined period of time, where after the predetermined period of time, the system can be configured to stop the flow of the at least one sample into the cartridge, and can also be configured to at least one of count parasites and oocysts;
the system is additionally configured to characterize counted parasites and/or oocysts;
each sample can be exposed to at least one fluorophore or fluorophore labeled agent, whereby exposure can be configured such that at least one of a nucleic acid intercalating dye, SYBR (or other DNA intercalating dye), fluorescent labeled lectins, fluorescent labeled antibodies, acid fast stains, membrane stains, and fluorophore labeled in-situ-hybridization probes can be visualized;
labeling of parasites and/or oocysts can occur prior to adding the sample to the cartridge, or, after capture in a capture region/zone;
characterizing captured parasites by at least one of size, shape. and other morphological parameter;
characterizing a level of sporulation of individual parasites contained in at least one sample;
determine a state of parasite sporulation;
visualizing a level of sporulation of individual parasites contained in at least one sample, such that sporulation can be visualized by exposing the at least one sample with at least one staining agent, the at least one staining agent may comprise a DNA intercalating dye, and wherein the DNA intercalating dye can comprise SYBR, or fluorophore labeled binding agent;
visualizing a level of sporulation of individual parasites contained in at least one sample, such that sporulation can be visualized in exciting the intrinsic autofluorescence of the parasites and imaging through an appropriate emission filter; and
the parasite and/or oocysts comprise Coccidia, wherein the coccidian can comprise at least one of genera Toxoplasma, Cryptosporidium, Cyclospora, Eimeria, and Cyclospora.

At least some of the embodiments disclosed herein (method, system, or device), can be configured for use in the formulation of vaccines, the assessment of the quality of vaccines, as well as, in some embodiments, to assess a size distribution of Eimeria oocysts in a vaccine preparation.

In some embodiments, a method for detecting parasitic infection in fecal samples in an assay system is provided, and includes preparing a plurality of fecal samples for analysis. Preparing comprises, for each sample labeling at least each of a first tube (e.g., 5 mL vortexing tube) and a second tube (e.g.,. 2.0 mL vortexing tube), placing a predetermined amount of sample (e.g., 0.4 g) within the first tube, placing a predetermined amount (e.g., 2 mL) of 1 M NaOH in the first tube, vortexing the first tube for a first predetermined period of time (e.g., 30 sec), placing a predetermined amount (e.g., 2 mL) of Sheather's sugar solution to the first tube, vortexing the first tube for a second predetermined period of time (e.g., 30 seconds), removing an amount of the sample from the first tube and determining a reference count of oocysts, transferring a third predetermined amount (e.g., 280 μL) of each fecal-NaOH mixture of each first tube into a respective second tube, adding a fourth predetermined amount (e.g., 20 μL) of ferrofluid and a fifth predetermined amount (e.g., 3 μL) of SYBR into each respective second tube, vortexing each respective second tube for a third predetermined amount of time (e.g., 5 seconds). Aliquots of samples prepared as described above are then added to individual lanes of an assay cartridge such as a Piper™ cartridge (Ancera LLC, or also referred to as a MagDrive™ cartridge). The cartridge is then placed into a Piper™ instrument and the sample is flowed into a lane wherein electromagnetic fields produced by the system and acting on the ferrofluid push up oocysts contained in the at least one sample to a zone where the oocysts are captured proximate a channel window provided for each channel, scanning each window of each channel to produce a respective scan image thereof, processing each respective scan image so as to at least one of identify and count each oocyst imaged from a respective channel window, converting each count from each processed image to oocysts per gram of original fecal sample, and optionally identifying and measuring the major axis of each counted oocyst such that the number of small (<18 μm), medium (18-25 μm), and large (>25 μm) oocysts are counted for each sample and optionally converted to oocysts per gram of original fecal sample. The extent of sporulation can also be determined from these images.

Such embodiments (as well as other embodiments) may further include one and/or another, and in some embodiments, a plurality of, and in some embodiments, a majority of, and in some embodiments, substantially all of, and in still further embodiments, all of (if not mutually exclusive), of the following steps, features, functionality, structure, and/or clarification, yielding yet further embodiments:
the metrics for small, medium, and large oocysts can use both major and minor axis to discriminate oocysts based on size; for example: >27 μm major axis=large; <27 μm major axis and >18 μm minor axis=medium; <27 μm major axis and <18 μm minor axis=small);
a sample can be prepared in a filter bag (e.g. Whirl-Pak filter bag) (e.g., instead of measuring a predetermined amount of sample in a tube) and after addition of a predetermined amount of 1 M NaOH (e.g. 5 ml), the sample is homogenized by massaging;
samples can be concentrated prior to analysis. For example, in one case, the concentration of Sheather's solution in a post homogenization step, can be raised to 1× (in some embodiments, oocysts have a density of approximately 1.11 g/mL and density of 1×Sheather's solution would be approximately 1.27 g/mL, thus, the oocysts/cells float in the 1×Sheather's solution. In some embodiments, oocysts/cells float in Sheather's solution when the solution density is greater than about 1.2 g/mL, although processing would be longer and required higher forces;
a sample can optionally be centrifuged at 2000 g for 3 minutes (for example). In either case, the process involves floating oocytes to the surface. The floating oocysts can then be removed, suspended to a set volume in water or a buffer, a sample is removed and labeled, mixed with ferrofluid and analyzed on an assay instrument device and/or system (e.g., Piper™);
and
a post homogenization sample can also be gently centrifuged at a relative centrifugal force of, e.g., 200 g for 1 minute to pellet the oocysts, but leave the fecal debris in suspension. The supernatant containing debris is then removed without disturbing the pellet of oocysts and then the pellet is resuspended to a set volume. An aliquot is then removed for analysis. Accordingly, in some embodiments, when there is no Sheather's solution, oocysts/cells will sink because they are denser than water, and thus, debris will pellet under centrifugation at a much slower rate (to this end, in some embodiments, it is possible to pellet the oocysts without pelleting all of the debris).

In one aspect, the present disclosure is direct to an assay system configured to at least one of detect, enumerate, and/or characterize one or more parasite oocysts from at least one fecal sample or a plurality of samples, comprising:

a ferrofluidic assay device configured to receive a ferrofluidic cartridge;

the ferrofluidic cartridge including a plurality of windows each adjacent a capture region configured to capture one or more predetermined parasite oocysts;

an imager configured to image each window of the cartridge either separately or together;

a controller configured to control at least one of the ferrofluidic assay device, the ferrofluidic cartridge, and the imager;

and

assay processing components comprising at least one of reagents, and controls

wherein the system is configured to at least one of: separate any and all of parasite oocysts or eggs and move or otherwise locate the parasite oocysts or eggs to one or more of the windows where they can be counted and characterized.

Such embodiments (as well as other embodiments) may further include one and/or another, and in some embodiments, a plurality of, and in some embodiments, a majority of, and in some embodiments, substantially all of, and in still further embodiments, all of (if not mutually exclusive), of the following steps, features, functionality, structure, and/or clarification, yielding yet further embodiments:
parasites oocysts or eggs contained within the plurality of samples are at least one of: labeled and visualized by fluorescence, and unlabeled and visualized by respective intrinsic auto-fluorescence;
the one or more parasite oocysts or eggs can being evaluated can be of the same genus or species of any parasite of interest;
the samples can be mixed with ferrofluid prior to being flowed;
   in some embodiments, mixed with ferrofluid prior to being flowed past at least one capture region;
the system or a device can establish at least one electromagnetic field to act on the ferrofluid so as to direct at least one of parasites oocysts or eggs contained in the sample to a specific area or location where they are counted and characterized;
the ferrofluid of the system can include at least one density modifying agents;
the ferrofluid can include at least one density modifying agents, where the at least one density modifying agent can comprise at least one of a concentrated sugar solution, and Sheather's solution;
and
each sample can at least one of be processed and mixed with sodium hydroxide. and/or detergents.

In some embodiments, at least one sample is processed and mixed with sodium hydroxide, wherein the sodium hydroxide is mixed with each sample by mixing equal volumes of sample and sodium hydroxide solution, mixing a 1:2 volume:volume of sample and sodium hydroxide solution, mixing a 1:3 volume:volume of sample and sodium hydroxide solution, mixing a 1:4 volume:volume of sample and sodium hydroxide solution, mixing a 1:5 volume:volume of sample and sodium hydroxide solution, mixing a 2:1 volume:volume of sample and sodium hydroxide solution, mixing a 3:1 volume:volume of sample and sodium hydroxide solution, mixing a 4:1 volume:volume of sample and sodium hydroxide solution, or mixing a 5:1 volume:volume of sample and sodium hydroxide solution, and optionally incubating the mixture prior to flowing the mixture over a capture region of the system.

In some embodiments, at least one sample is processed and mixed with sodium hydroxide, wherein the concentration of sodium hydroxide is: between 0.01 molar (M) and 2M, between 0.2M and 1.2M, at about 0.2M to about 1M, or at about 1M.

In some embodiments, the mixture is incubated.

In some embodiments, the mixture is incubated at about room temperature. In some embodiments, the mixture is incubated at between 15-25° C. In some embodiments, the mixture is incubated at between 18-20° C. In some embodiments, the mixture is incubated at 20° C. In some embodiments, the mixture is incubated at 25° C.

In some embodiments, the mixture is incubated for less than 45 minutes, between 1-45 minutes, less than 60 minutes, between 1-60 minutes, about 15 minutes, about 30 minutes, about 1 hour, about 2 hours, about 3 hours, about 4 hours, about 5 hours, about 6 hours, about 12 hours, or between 1-24 hours prior to the samples being analyzed.

In some embodiments, the samples are added to a ferrofluid. In some embodiments, the samples are added to ferrofluid at a predetermined ratio, e.g., 1 part ferrofluid to 14 parts sample, 1 part ferrofluid to 10 parts sample, 1 part ferrofluid to 9 parts sample, 1 part ferrofluid to 8 parts sample, 1 part ferrofluid to 7 parts sample, 1 part ferrofluid to 6 parts sample, 1 part ferrofluid to 5 parts sample, 1 part ferrofluid to 4 parts sample, 1 part ferrofluid to 3 parts sample, 1 part ferrofluid to 2 parts sample, 1 part ferrofluid to 1 part sample, or 2 parts ferrofluid to 1 part sample.

In some embodiments, the system of the present disclosure further comprises at least one of a hemocytometer and a McMaster chamber.

In some embodiments, the system of the present disclosure further comprises at least one of a hemocytometer and McMaster chamber, where at least one of the hemocytometer and McMaster chamber are configured to cause fluorescence excitation/emission of fluorophore labeled parasite oocysts or eggs in the samples using wavelengths specific to the fluorophore used.

In some embodiments, cells are counted without being captured.

In some embodiments, each capture region is coated with a binding agent configured to bind with surface features on at least one of the parasite oocysts or eggs.

In some embodiments, the binding agent comprises at least one of an antibody, aptamer, lectin, and mucin.

In some embodiments, the binding agent is bound to the capture region via at least one of avidin, streptavidin, neutravidin, and any other modified binder.

In some embodiments, the system is configured to flow at least one of the plurality of samples into cartridge for a predetermined period of time.

In some embodiments, after a predetermined period of time, the system is configured to stop the flow of the at least one sample into the cartridge, and the captured parasite oocysts or eggs are counted.

In some embodiments, the system of the present disclosure is additionally configured to characterize the counted parasite oocysts or eggs.

In some embodiments, each sample is exposed to at least one fluorophore or fluorophore labeled agent.

In some embodiments, exposing each sample to at least one fluorophore or fluorophore labeled agent is configured such that at least one of SYBR, fluorescent labeled lectins, fluorescent labeled antibodies, acid fast stains, membrane stains, and fluorophore labeled in-situ-hybridization probes can be visualized.

In some embodiments, parasite oocysts or eggs are labeled after capture in a capture region/zone.

In some embodiments, parasite oocysts or eggs are pre-labeled prior to processing.

In some embodiments, the system is configured to characterize captured oocysts or eggs by at least one of size, shape, and other morphological parameter.

In some embodiments, the system is further configured to characterize a level of sporulation of individual oocysts contained in at least one sample.

In some embodiments, the system is further configured to determine a state of oocyst sporulation.

In some embodiments, the system is further configured to visualize a level of sporulation of individual oocysts contained in at least one sample.

In some embodiments, sporulation is visualized by exposing the at least one sample with at least one staining agent, the at least one staining agent may comprise a DNA intercalating dye, and wherein the DNA intercalating dye can comprise SYBR.

In some embodiments, the parasite oocysts or eggs comprise Coccidia.

In some embodiments, the coccidia comprises at least one of genera Toxoplasma, Cryptosporidium, Cyclospora, Eimeria, and Cyclospora.

In some embodiments, the present disclosure is directed to a method, system or device as described herein, further configured to assess a size distribution of Eimeria in vaccine preparation.

In one aspect, the present disclosure is directed to a method for detecting parasitic infection in fecal samples in an assay system comprising:

preparing a plurality of fecal samples for analysis, wherein preparing comprises, for each sample:

labeling at least each of a first tube (e.g., 5 mL vortexing tube or 10 mL vortexing tube) and a second tube (e.g., 2.0 mL vortexing tube or 1.7 ml vortexing tube)

placing a predetermined amount of sample (e.g., 0.1 g, 0.2 g, 0.3 g, 0.4 g, 0.5 g, 0.6 g, 0.7 g, 0.8 g, 0.9 g, 1 g, or between 1 g and 5 g) within the first tube;

placing a predetermined amount (e.g., 1 mL, 2 mL, 3 mL, 4 mL, 5 mL, or between 1 mL and 5 mL) of NaOH at a pre-determined concentration (e.g. 0.5M, 0.6M, 0.7M, 0.8M, 0.9M, or 1M) in the first tube;

vortexing the first tube for a first predetermined period of time (e.g., 15 sec, 30 sec, 45 sec, 1 min, or between 15 and 60 seconds);

placing a predetermined amount (e.g., 1 mL, 2 mL, 3 mL, 4 mL, 5 mL, or between 1 mL and 5 mL) of Sheather's sugar solution to the first tube;

vortexing the first tube for a second predetermined period of time (e.g., 15 sec, 30 sec, 45 sec, 1 min, or between 15 and 60 seconds);

optionally removing an amount of the sample from the first tube and determining a reference count of oocysts;

transferring a third predetermined amount (e.g., 200 μL, 210 μL, 220 μL, 230 μL 240 μL, 250 μL, 260 μL, 270 μL, 280 μL, 290 μL, 300 μL, between 200-300 μL, or between 260-300 μL) of each fecal-NaOH mixture of each first tube into a respective second tube;

adding a fourth predetermined amount (e.g., 10 μL 20 μL, 30 μL, 40 μL, 50 μL, 60 μL or between 10-60 μL) of ferrofluid and a fifth predetermined amount (e.g., 3 μL or between 1-5 μL) of SYBR into each respective second tube;

vortexing each respective second tube for a third predetermined amount of time (e.g., 15 sec, 30 sec, 45 sec, 1 min, or between 15 and 60 seconds);

add 250 μl or 300 μl of the vortexed samples to lanes of an assay cartridge having a plurality of channels/lanes, each channel configured with at least one capture region/zone having at least one unique agent for binding with at least one specific parasite oocyst or egg;

flowing the mixture over at least one of the channels of the cartridge, wherein electromagnetic fields produced by the system and acting on the ferrofluid push up oocysts contained in the at least one sample to a capture region proximate a channel window provided for each channel;

scanning each window of each channel to produce a respective scan image thereof;

processing each respective scan image so as to at least one of identify and count each oocyst imaged from a respective channel window;

converting each count from each processed image to oocysts per gram of original fecal sample; and

optionally identifying and measuring the major axis of each counted oocyst such that the number of small (<18 μm), medium (18-25 μm), and large (>25 μm) oocysts are counted for each sample and optionally converted to oocysts per gram of original fecal sample.

In one aspect, the present disclosure is directed to a method for detecting parasitic infection in fecal samples in an assay system comprising:

placing a predetermined amount of animal fecal matter into a first side (“fecal side”) of at least a two-sided sample filter bag (“sample bag”);

adding a predetermined amount of NaOH to the first side of the sample bag forming a first mixture;

performing a first homogenization of the mixture, via, for example, massaging the mixture for a first predetermined period of time;

incubating the mixture for a second predetermined period of time;

adding in a predetermined amount of Sheather's solution to a second side of the sample bag (“filtered side”);

performing a second homogenization of the mixture, via, for example, massaging the mixture of a second predetermined period of time;

removing an aliquot of a predetermined amount from the filtered side of the bag;

transferring the aliquot to a tube of predetermined size;

adding a predetermined amount of ferrofluid to the tube;

vortexing the tube for a predetermined period of time;

transferring the contents of the tube to a single sample well of the assay cartridge;

flowing each sample from each sample well into an imaging window:

push all cells to the surface of the imaging zone;

optionally repeating the above-noted steps a plurality of times so as to utilize a corresponding number of sample wells, such that, each sample corresponds to a unique sample well.

optionally flowing each sample from each sample well into at least one imaging/capture zone of the assay cartridge so as to capture at least one specific parasite oocyst or egg contained in the sample;

acquiring image of each imaging zone;

determining a number of parasite oocysts or eggs (e.g., coccidian) present in the imaging zone; and

determining a size distribution of the parasite oocysts or eggs captured in the at least one imaging zone.

In some embodiments, the method further comprises adding in a predetermined amount a fluorophore or fluorophore labeled agent to the tube.

In some embodiments, the parasite oocysts or eggs are labeled with at least one fluorophore or fluorophore labeled agent, wherein the fluorophore or fluorophore labeled agent is selected from the group consisting of SYBR, fluorescent labeled lectins, fluorescent labeled antibodies, acid fast stains, membrane stains, or fluorophore labeled in-situ-hybridization probes.

In some embodiments, the fluorophore or fluorophore labeled agent is added prior to mixing the sample with ferrofluid.

In some embodiments, no fluorescent labeling agent is used, and the parasite oocysts or eggs are monitored by their intrinsic fluorescence.

In some embodiments, the predetermined amount of animal fecal matter is between 0.1 g and 5 g.

In some embodiments, the predetermined amount of NaOH is 1 mL, 2 mL, 3 mL, 4 mL, 5 mL, or is between 1 mL and 5 mL and the concentration of NaOH is 0.1M, 0.2M, 0.3M, 0.4M, 0.5M, 0.6M, 0.7M, 0.8M, 0.9M, 1M, is between 0.1M and 2M, is between 0.1M and 1M.

In some embodiments, the first predetermined period of time is 30 seconds, 1 minute, 5 minutes, 10 minutes, 15 minutes, 30 minutes, or between 30 seconds and 2 minutes, between 1-15 minutes, or between 1 and 30 minutes.

In some embodiments, the second predetermined period of time is less than 30 minutes, 1-30 minutes, less than 45 minutes, between 1-45 minutes, less than 60 minutes, between 1-60 minutes, about 15 minutes, about 30 minutes, or about 1 hour.

In some embodiments, the predetermined amount of Sheather's sugar solution is 1 mL, 2 mL, 3 mL, 4 mL, 5 mL, or is between 1 mL and 5 mL.

In some embodiments, the second predetermined period of time is 30 seconds, 1 minute, 5 minutes, 10 minutes, 15 minutes, 30 minutes, or between 30 seconds and 2 minutes, between 1-15 minutes, or between 1 and 30 minutes.

In some embodiments, the aliquot of a predetermined amount is 200 μL, 210 μL, 220 μL, 230 μL, 240 μL, 250 μL, 260 μL, 270 μL, 280 μL, 290 μL, 300 μL, between 200-300 μL, or between 260-300 μL.

In some embodiments, the tube of predetermined size is a 1.7 mL to 2 mL tube.

In some embodiments, predetermined amount of ferrofluid added to the tube is 10 μL 20 μL, 30 μL, 40 μL, 50 μL, 60 μL or between 10-60 μL.

In some embodiments, predetermined amount of concentrated SYBR to the tube is 1 μL, 2 μL, 3 μL, 4 μL, 5 μL or between 1-5 μL.

In some embodiments, the method is performed using a microfluidic device.

In some embodiments, the method is performed using a ferrofluid-based microfluidic device.

In some embodiments, the parasite cells or oocysts are counted using at least one of a hemocytometer and McMaster chamber.

In some embodiments, at least one of the hemocytometer and McMaster chamber are configured to cause fluorescence excitation/emission of fluorophore labeled parasites and/or oocysts in the samples using wavelengths specific to the fluorophore used.

In some embodiments, the method further comprises flowing at least one of the plurality of samples into cartridge for a predetermined period of time.

In some embodiments, the method further comprises counting the parasite oocysts or eggs without capture.

In some embodiments, the method further comprises characterizing the counted parasite oocysts or eggs.

In some embodiments, wherein the method further comprises characterizing a level of sporulation of individual oocysts or eggs

contained in at least one sample.

In some embodiments, the method further comprises determining a state of parasite oocyst or egg sporulation.

In some embodiments, the method further comprises visualizing a level of sporulation of individual parasite oocysts or eggs contained in at least one sample.

In some embodiments, sporulation is visualized by exposing the at least one sample with at least one staining agent, the at least one staining agent may comprise a DNA intercalating dye, and wherein the DNA intercalating dye can comprise SYBR.

In some embodiments, the method, system or device discussed herein is further configured to assess a size distribution of Eimeria oocysts in vaccine preparation.

### BRIEF DESCRIPTION OF DRAWINGS

FIG. 1 shows a hemocytometer image of a feces samples with NaOH treatment of Coccivac-B52 oocysts, according to embodiments of the present disclosure.

FIG. 2 shows a hemocytometer image of a feces samples without NaOH treatment.

FIG. 3 is an image of oocysts captured on ConA-coated slide, pretreated with chloroform-methanol, according to some embodiments of the disclosure.

FIGS. 4A and 4B show the data and diagram from settling experiments, where four fecal sub-samples were prepared with either 1:10 feces: NaOH or 1:5:5 feces:NaOH: Sheather's and then sampled at different levels in the tube either after vortexing or after 5 minutes of settling. Specifically, FIG. 4A shows average hemocytometer oocyst counts of sample mixed with 1 g feces to 10 mL 1M NaOH (NaOH only), and FIG. 4B shows a 1 g fecal sample mixed with 5 mL NaOH and 5 mL Sheather's sugar solution. Each aliquot corresponds with the aliquots as described in FIG. 5.

FIG. 5 is an illustration of sample aliquots tested in oocyst experiments according to some embodiments of the disclosure.

FIG. 6 shows an image of four (4) species of Eimeria oocysts fluorescently labeled with SYBR Green, in ferrofluid and NaOH mixture. Note the labeling of the inner structures, i.e., label A=oocyst with unsporulated cytoplasmic mass, and the other ovoid shapes show labeled sporocysts). The Image obtained on PIPER™ instrument with 300 ms exposure.

FIG. 7 shows a fecal sample fluorescently labeled with MPL with conjugated AlexaFfluor488 (image acquired on PIPER™, in the presence of ferrofluid).

FIG. 8 shows a ConA-coated cartridge showing bound oocysts fluorescently labeled with SYBR. The blue squares loosely mark the coated window, where the ConA is coated onto the cartridge and where oocysts are bound. FIG. 8, left, which is a 100 ms exposures of bound SYBR-stained oocysts. FIG. 8, right shows the same region with a 300 ms exposure after a 5-minute flush of the cartridge, showing oocysts still bound in place.

FIGS. 9A and 9B are images of the same corner of the ConA coated cartridge images shown in FIG. 8.

FIG. 10 illustrates Auramine O fluorescently labeled oocysts in the presence of ferrofluid.

FIG. 11 illustrates oocysts fluorescently labeled with MitoTracker Green.

FIG. 12 illustrates a sample labeled with SYBR without NAOH present (e.g., see above) in a lane (including an imaging window) of a processing assay (e.g., cartridge).

FIG. 13 shows a schematic of Assay Workflow. FIG. 13A: Sample preparation. 1 g of a fecal sample is mixed with 5 ml of 1M NaOH in a filter bag (side A). The bag is massaged for 30 seconds to thoroughly mix the sample followed by incubation for 15 minutes at room temperature. Sheather's solution is subsequently added to a final concentration of 50% to prevent oocyst settling, and the sample is mixed again. A 280 μl aliquot of the slurry is then removed from the filtered side of the bag (side B) to avoid any solids that could clog the microfluidic device and transferred to a new tube. The sample is mixed with 20 μl of ferrofluid and 3 μl of SYBR Green stain, vortexed to mix, and loaded into a single well of a PIPER™ cartridge. FIG. 13B: Separation of oocysts on PIPER™. Sample is mixed with a biocompatible ferrofluid and loaded into a cartridge. Microvalves in the cartridge control a pumping layer in the cartridge which pulls the sample over the magnetic PCB, which pushes target cells up for imaging by a built-in epifluorescent microscope above the cartridge. Data can be transferred to a cloud-based system or to a connected laboratory information management system based on the needs of the end user.

FIG. 13C is a perspective view of an assay cartridge for use in the assay device for the assay system of FIG. 13B, according to some embodiments of the disclosure.

FIG. 14. Example of PIPER™ image detecting oocysts. Image is magnified 100%. Detected oocysts are indicated by circles. Color discriminates oocysts based on size: large (yellow), medium (blue), or small (green).

FIG. 15 is a plot of counts obtained by manual review of 67 images (corresponding to replicates from 3 individual, fecal samples) to counts obtained for the same images by the image recognition algorithm. A plot of manual counts to algorithm counts for each of the images shows a slope near 1 and r2 value of 0.99.

FIG. 16 shows paired hemocytometer and PIPER™ data were plotted against each other for 77 independent samples to determine the calibration of PIPER™ counts to oocysts per gram (OPG). The plot had a linear curve fit with a calculated coefficient of determination (R-squared) of 0.9835.

FIG. 17 shows a graph of the average log hemocytometer OPG versus the CV of hemocytometer (orange) or PIPER™ (teal) counts. The line is a LOESS local regression used to generate a smoothed curve representing each CV. The shadows represent the standard error (95% confidence interval) of the mean CV of each measurement. The vertical gray line demarcates 2 million OPG.

FIG. 18 is a plot of PIPER™ counts obtained for 3 different amounts of each of four, cleaned oocyst samples versus the predicted counts for each sample portion. Replicates (3 or 4 as indicated on the graph) were obtained for each portion of each sample. The average count for the 1× portion of each sample was multiplied by 0.3 or 0.1, respectively, to calculate a predicted count for the smaller portions. The r2 value for the line y=x was 0.9666.

FIG. 19 is a plot of the relative percent difference between PIPER™ and hemocytometer. Squares, triangles, and circles indicated three sites of analysis. Average counts for the replicates of each sample were calculated. The relative percent difference was computed using the formula RPD=[2*(hemocytometer OPG−average PIPER™ OPG)]/[(hemocytometer OPG+PIPER™ OPG)/2]. The dashed lines at +/−66.6% represent a two-fold difference.

### DETAILED DESCRIPTION

While various inventive embodiments have been described and illustrated herein, those of ordinary skill in the art will readily envision a variety of other means, functionality, steps, and/or structures (including software code) for performing the functionality disclosed and/or obtaining the results and/or one or more of the advantages described herein, and each of such variations and/or modifications is deemed to be within the scope of the inventive embodiments described herein. More generally, those skilled in the art will readily appreciate that all parameters, and configurations described herein are meant to be exemplary and that the actual parameters, and configurations will depend upon the specific application or applications for which the inventive teachings is/are used. Those skilled in the art will recognize, or be able to ascertain using no more than routine experimentation, many equivalents to the specific inventive embodiments described herein. It is therefore to be understood that the foregoing embodiments are presented by way of example only and that, within the scope of any claims supported by this disclosure and equivalents thereto, inventive embodiments may be practiced otherwise than as specifically described and claimed. Inventive embodiments of the present disclosure are also directed to each individual feature, system, apparatus, device, step, code, functionality and/or method described herein. In addition, any combination of two or more such features, systems, apparatuses, devices, steps, code, functionalities, and/or methods, if such features, systems, apparatuses, devices, steps, code, functionalities, and/or methods are not mutually inconsistent, is included within the inventive scope of the present disclosure. Further embodiments may be patentable over prior art by specifically lacking one or more features/functionality/steps (i.e., claims directed to such embodiments may include one or more negative limitations to distinguish such claims from prior art).

The embodiments of the present disclosure can be implemented in any of numerous ways. For example, some embodiments may be implemented (e.g., as noted) using hardware, software or a combination thereof. When any aspect of an embodiment is implemented at least in part in software, the software code can be executed on any suitable processor or collection of processors, servers, and the like, whether provided in a single computer or distributed among multiple computers.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Provided herein are integrated, automated diagnostic assays systems which may be used to detect, enumerate, and/or characterize parasites or oocysts in fecal samples.

The system may be a fluidic system, such as a ferrofluidic system.

The systems provided herein may be used to detect the parasites directly, or to detect oocysts produced by the parasites. Any parasite or parasite oocyst may be analyzed using the system provided herein. For example, parasites of the Emeria species (e.g., E. acervulina, E. tenella, or E. maxima), may be detected, enumerated and/or characterized using the systems and methods described herein.

The systems and methods described herein may be used to determine the number or concentration of parasites in a sample, or to determine the state of parasite sporulation. The methods may be used to diagnose an animal suspected to be infected with the parasite. For example, the methods may be used to identify flocks of poultry that are at risk of decreased performance levels due to parasite infection, and help in the development of treatment strategies.

The methods may also be used to assess the quality of vaccines by determining contamination with parasites.

At least some of the structure for one and/or another of the embodiments can be found in one or more of the following disclosures: The current disclosure is related to U.S. Pat. Nos. 8,961,878, and 9,999,855, and PCT publication nos. WO2014144340, WO2014144782, WO2014144810, WO2014145765, WO2014165317, WO2016210348, WO2017004595, WO2018026605, WO2019117877, and WO2022/015845.

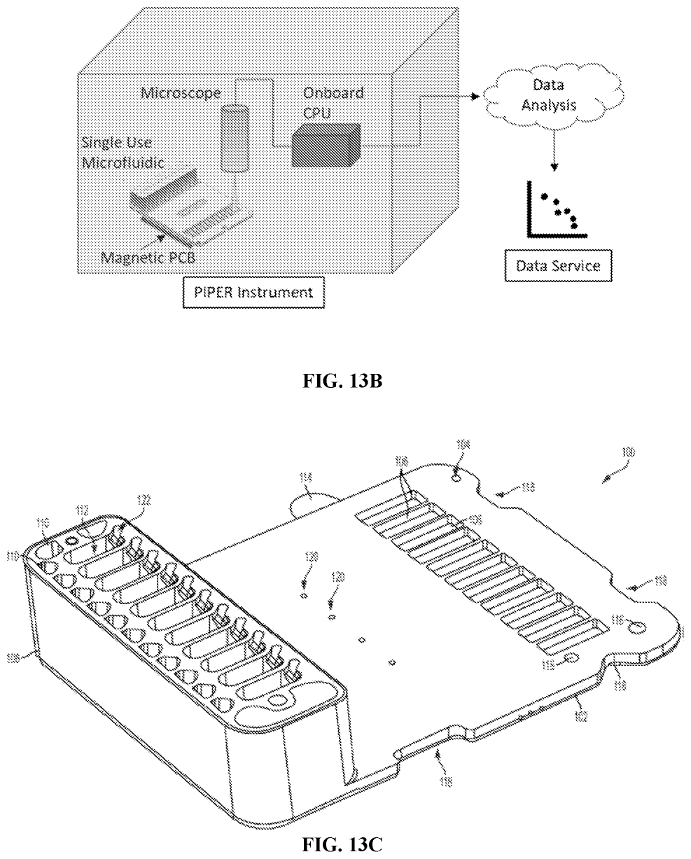

While the published PCT disclosures incorporated herein disclose various systems, devices and methods for performing ferrofluidic assays for use with embodiments of the present disclosure, FIGS. 13A-13C illustrate embodiments of exemplary assay systems. As shown, FIG. 13B illustrates a ferrofluidic assay system 130 including a ferrofluidic assay device 132 configured to receive a ferrofluidic cartridge 134 (e.g., Piper™ cartridge) in a receiving area. The ferrofluidic cartridge 134 includes a plurality of windows (see FIG. 13C) each adjacent a capture region configured to capture one or more predetermined parasite oocysts or eggs, an imager 136 (e.g., microscope(s)) configured to image each window of the cartridge either separately or together, a controller/processor/CPU 138 configured to control at least one of the ferrofluidic assay device, the ferrofluidic cartridge, and the imager, and assay processing components comprising at least one of reagents, and controls (not shown). The system is configured to at least one of: separate any and all of oocysts or eggs and move or otherwise locate the oocysts or eggs to one or more of the windows. The data produced by the ferrofluidic assay system can be analyzed either on the assay device (e.g., using the processor), or via a data service 140.

Assay cartridges, for use in assay systems according to some embodiments, can include those as set out in published PCT application WO2018/026605 (incorporated herein by reference), as well as similar cartridges thereto. FIG. 20 illustrates a perspective view of a cartridge that is configured to perform multiple, independent, parallel assays (e.g., 2-10, 2-20, 2-100 or more, and ranges therebetween). Accordingly, the width of the cartridge 100 may change depending on the total number of assays supported.

As shown in FIG. 13C, assay cartridge 100 may comprise multiple layers integrated into a unitary/integral or an integrated cartridge (e.g., cartridge 100 may comprise a single construction with various features discussed below integrated therein). Cartridge 100 may include base layer 102, cartridge-instrument alignment features 118, a reagent spotting mask 114, pump valves 120 and a reservoir stack 108. Reservoir stack 108 may further include main reservoirs 112 (which can contain samples or mixtures of samples, regents, and the like), return chimneys 122 and a plurality of secondary (and, in some implementations, tertiary, etc.) reservoirs 110. The cartridge may also comprise internal alignment features 104 and 116 that may be used to ensure proper registration between the internal layers during its construction.

Cartridge-instrument alignment features 118 enable aligning placement of cartridge 100 within an assay instrument (not shown). The alignment may ensure, in part, that the cartridge main channels can align directly (or approximately) over the electrodes of an excitation PCB. This may also ensure that any other interface to the cartridge (such as pneumatic input ports for pumping fluid reagents within the cartridge) are aligned with the corresponding output from the instrument. Cartridge 100 may be inserted into an instrument slot (not shown) or may be placed at a designated space (such as a dedicated receptacle) within the assay instrument (not shown).

A plurality of cartridge analysis windows (or viewing ports) 106 are arranged to correspond with each of a plurality of reaction channels (not shown). The reaction channels within the cartridge may be embedded or formed over base 102. Cartridge analysis windows 106 provide optical viewing ports to each of the reaction channels.

The reagent spotting mask 114 may optionally be added to accommodate, for example, the precise positioning and spotting of assay reagents (e.g., capture reagents such as antibodies, aptamers, DNA fragments, other proteins or molecules used for surface modification or detection, etc.). The mask may consist of a matrix of patterned openings over an adhesive or a soft gasket (e.g., silicone rubber, PDMS, etc.) that is temporarily affixed over one of the bounding surfaces of the main assay channels. The assay reagents may thus be coated (or spotted) over that surface of the cartridge through the mask openings, either during the assembly of the cartridge or prior to running the assay by the end-user. Following an optional incubation period, the coated (or spotted) windows might be washed and/or dried, and the reagent spotting mask 114 may be removed (e.g., peeled off the cartridge surface) prior to capping the main assay channels with the final capping layer of the multi-stack assembly.

The internal alignment features 104 and 116 may optionally be used to assist in the assembly of the cartridge internal layers in order to ensure that each layer is properly aligned with and registered to its neighbors within a given positional tolerance. In some embodiments, the alignment features may be holes of a given shape (e.g., circular, square, hexagonal, diamond, etc.) that mate with alignment posts on an alignment jig.

The cartridge may have pneumatic input ports 120 which lead into pneumatic lines integrated into the cartridge. Together, they relay pressure and/or vacuum signals from the instrument to membrane valves (not shown) integrated into the body of the cartridge.

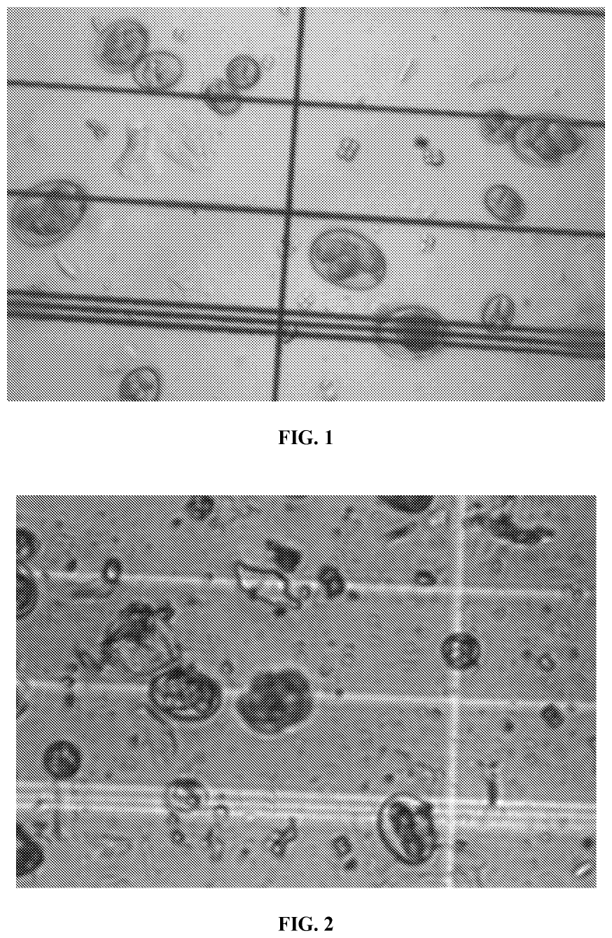

Reservoir stack 108 can retain the cartridge input fluids. For example, the reservoir stack 108 may receive and retain assay reagents which are then directed to the fluidic network (not shown in FIG. 1) of cartridge 100. Main reservoirs 112 typically receive ferrofluid and/or input sample reagents that are intended for the ferrofluidic assay. They may also be configured to receive additional reagents, as needed.

Reservoir stack 108 may support more than one set of reservoir wells per independent assay. Secondary reservoirs 110 may be configured to receive secondary reagents used for an assay under study. The secondary reagents may include labels, dyes, secondary antibodies, PCR reagents required for DNA amplification after cell capture, etc. In some implementations, the secondary reservoirs may be left blank or empty.

Some of the embodiments of the present disclosure are evident by reference to the following examples.

### EXAMPLE 1

1 g of feces was combined with 10 mL of 1M NaOH at room temperature, with no incubation time, prior to adding other assay components. This provided both increased binding to coated surfaces, as well as, unexpectedly, enabled the removal of debris present in the sample (e.g., see FIGS. 1 and 2). Fecal samples homogenized in 1M NaOH treatment at 20-38° C. for between 0-60 minutes, which provides cleaner labeling of oocysts, improved binding by lectins, and reduced fecal debris in the sample.

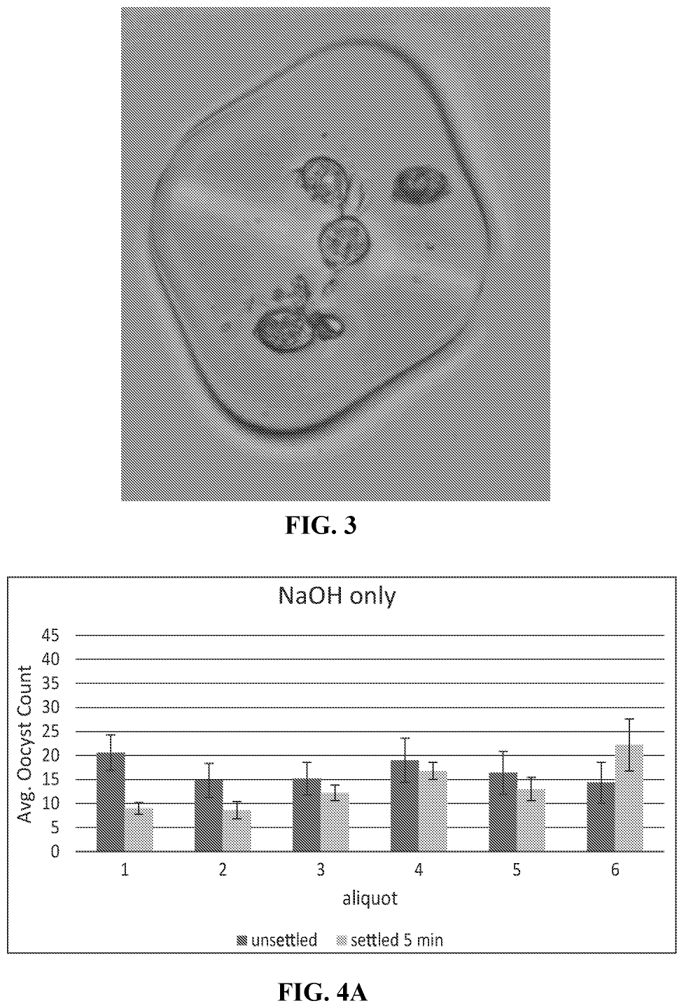

Chloroform-methanol (2:1) pre-treatment of fecal samples was also assessed. Accordingly, samples were treated with chloroform-methanol at 38° C. for 60-240 minutes. This treatment led to increased exposure of the oocyst wall structures and binding of the oocyst wall by lectins. See, e.g., FIG. 3, which is an image of oocysts captured on ConA-coated slide, pretreated with chloroform-methanol.

### EXAMPLE 2

1 gram of poultry feces was placed into one side of a Whirl-Pak filter bag. 5 mL of 1N NaOH was then added to the same side of the bag as the feces and the sample was homogenized by massaging the sample for 1 minute. After incubation for 15 minutes at room temperature, 5 mL of Sheather's solution was added to the opposite side of the filter bag. The sample was further massaged for 1 minute. A 280 mL aliquot was removed from the filtered (non-feces) side of the bag and transferred to a 2 mL tube.

Thereafter, 20 mL of stock ferrofluid and 3 mL of concentrated SYBR was added to the tube. The tube was then mixed by vortexing, and the entire volume was transferred to one sample well of a cartridge (e.g., an Ancera LLC Piper™ cartridge, which can be done for 12 samples to fill one cartridge). The cartridge is then run on an assay system or device (e.g., an Ancera LLC Piper™ instrument, where the sample is flowed into an imaging zone and multiple images are acquired and assembled into a larger image. Both the number of coccidia present and the size distribution of the coccidia are determined and reported.

Settling and Distributions. When mixed into a slurry with anything other than a high-salt or high-sugar solution (e.g. Fecasol or Sheather's Solution, respectively), oocysts naturally settle to the bottom of the container. With the high specific gravity solutions (e.g., >1.2), oocysts tend to float to the top of the container. The density of the solution can be adjusted to minimize the settling or flotation of the oocyst in the sample (according to some embodiments). Accordingly, to provide a more uniform sample without the concern of sampling affecting the counts, the samples were mixed as follows: 1 part feces, 5 parts 1M NaOH ratio, and 5 parts of a 1.27 specific gravity sugar solution. This prevents, or in some embodiments, lessens the settling of oocysts and provides a more uniform distribution of oocysts throughout the sample.

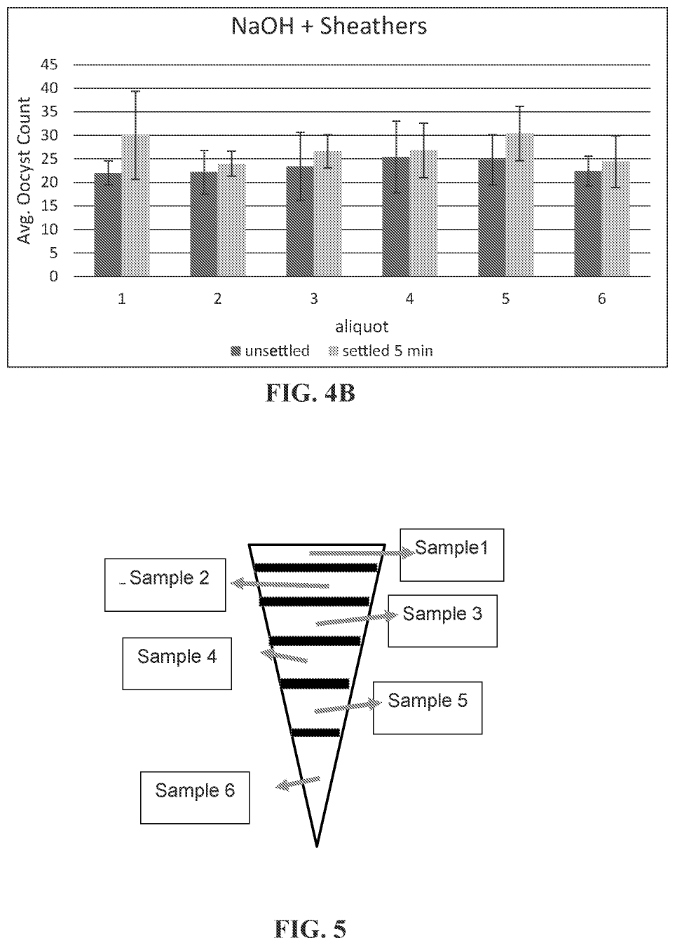

FIGS. 4a-b and 5 show the data and diagram from settling experiments, where four fecal sub-samples were prepared with either 1:10 feces: NaOH or 1:5:5 feces:NaOH:Sheather's and then sampled at different levels in the tube either after vortexing or after 5 minutes of settling. Specifically, FIG. 4a shows average hemocytometer oocyst counts of sample mixed with 1 g feces to 10 mL 1M NaOH (NaOH only), and FIG. 4b shows a 1 g fecal sample mixed with 5 mL NaOH and 5 mL Sheather's sugar solution. Each aliquot corresponds with the aliquots as described in FIG. 5.

The addition of sugar solution to the NaOH and feces creates a significantly more uniform sample. Additionally, using wide-bore pipette tips to transfer samples decreases the variability seen due to pipetting clogs and errors

In some embodiments, filter bags, such as those available from Whirl-Pak can also be used to lessen the effect of debris on pipetting. These bags are divided into 2 sections by a mesh filter. In some current embodiments, the feces sample plus NaOH and aqueous buffer is placed in the bag on one side of the filter, and additional buffer, or Sheather's solution is added to the opposite side of the filter. These bags facilitate homogenization of samples and mixing of reagents, and ease sample removement from the filtered (non-feces) side of the bag.

Labeling. Most conventional methods for Eimeria detection rely on light microscopy (2004 Holdsworth et al.). Flow cytometry has also been used for detection and identification (1989 Fuller and McDougald) of Eimeria. The PIPER™ instrument see above, leverages the use of ferrofluid for cell/particle sorting. The oocysts must fluoresce in order to be visible in the black background of the ferrous oxide nanoparticles. Such fluorescence can be obtained via use of at least one of: intercalating dyes, mitochondrial stains, labeled antibodies or antibody like binders, and lectins of various types, which have been proven to fluorescently label oocysts in the presence of ferrofluid.

In some cases, the intrinsic autofluorescence of the Eimeria membranes is sufficient for image processing.

Cyanine Dyes. Cyanine dyes such as SYBR Green are known for their capability to intercalate with and fluorescently label dsDNA (2005 Biver et al.), and can interact with different biological molecules through either covalent or noncovalent bonding, including brightly fluorescing when intercalating with DNA and interactions with the minor groove and creation of chiral aggregates of the dyes starting from nucleic acid templates (Armitage 2005). These dyes have not been described as oocyst labels in any genus. SYBR Green and SYBR Gold have been used in PCR-based detection assays (2008 Kawahara et al., 2002 Tanriverdi et al.).

Utilizing SYBR Green, the oocyst walls and some of their inner structures are fluorescently labeled, and labeling of the oocysts is near-instantaneous and the oocysts can be visualized by fluorescent microscopy after adding minute amounts of the dye. This fluorescent labeling also works in the presence of ferrofluid, 1M NaOH, and mixed sugar and NaOH solutions as previously described. Accordingly, while the high pH of the NaOH solution significantly quenches the fluorescence intensity of SYBR, there is sufficient signal to visualize the oocysts as well as some internal structure.

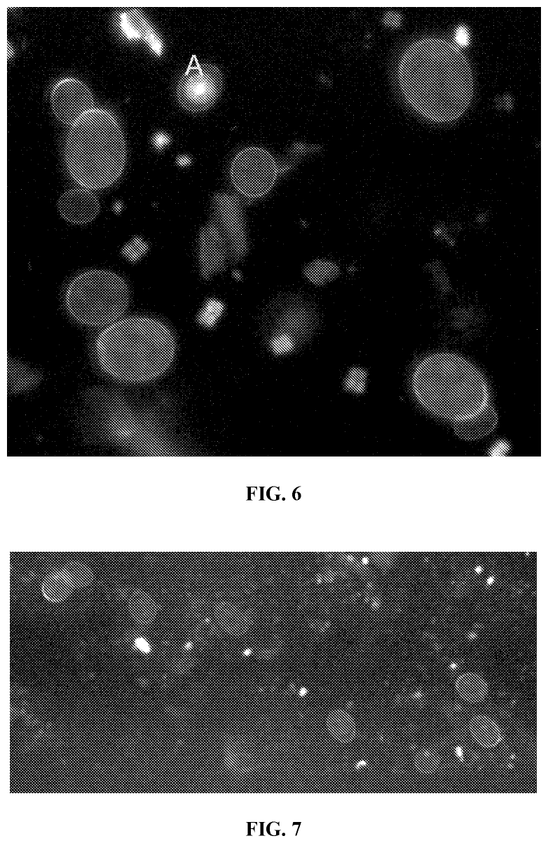

FIG. 6 shows an image of four (4) species of Eimeria oocysts fluorescently labeled with SYBR Green, in ferrofluid and NaOH mixture. Note the labeling of the inner structures, i.e., label A=oocyst with unsporulated cytoplasmic mass, and the other ovoid shapes show labeled sporocysts). The Image obtained on PIPER™ instrument with 300 ms exposure.

Lectins—Label and Capture Agent. From articles describing the biochemical structure of oocyst walls (Belli et al. 2006, Bushkin et al. 2013), structural components of the outer oocyst wall include beta-glucan fibrils, which can be bound to lectins. Binding of lectins with beta glucans, wheat germ agglutinin (WGA), soybean agglutinin (SBA), concanavalin A (ConA), peanut agglutinin (PNA), dectin-1, and Maclura pomifera lectin (MPL) were assessed.

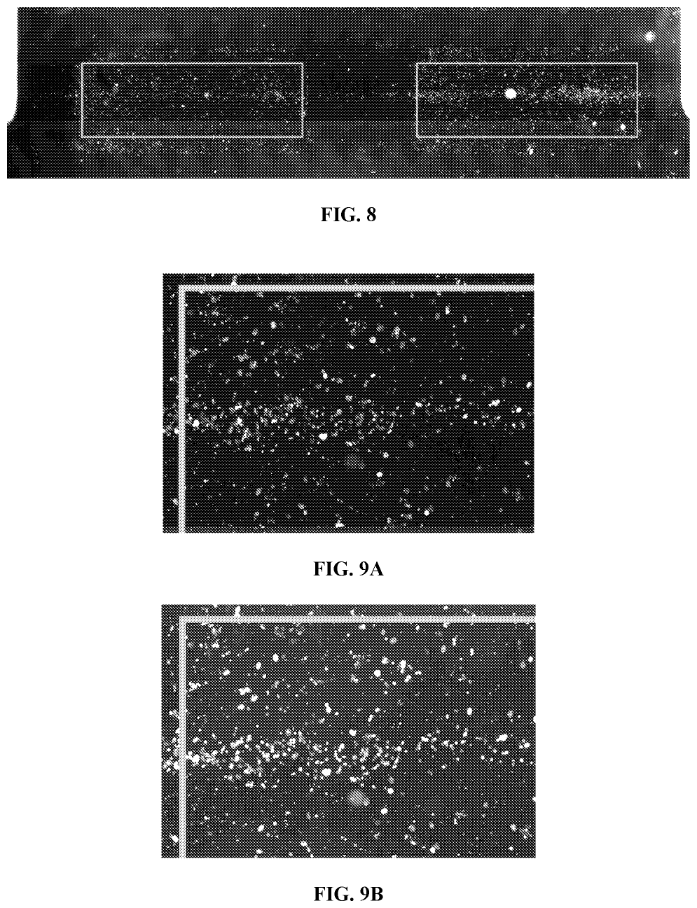

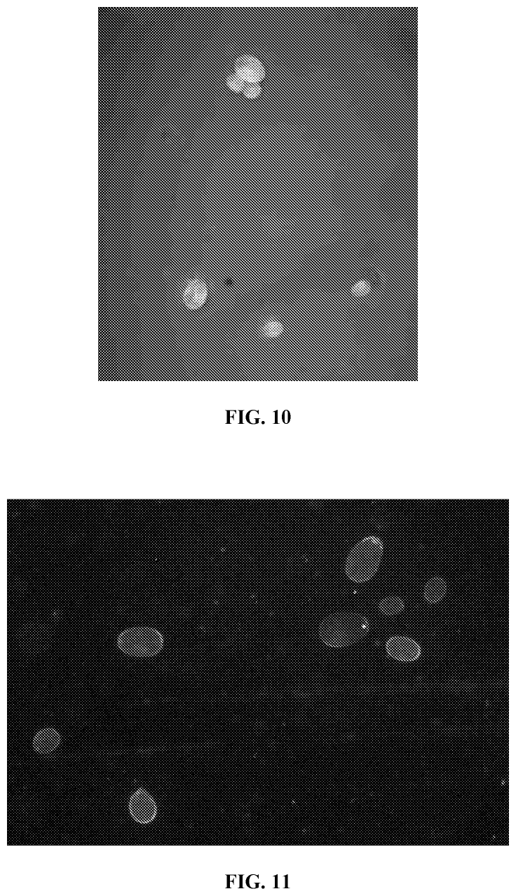

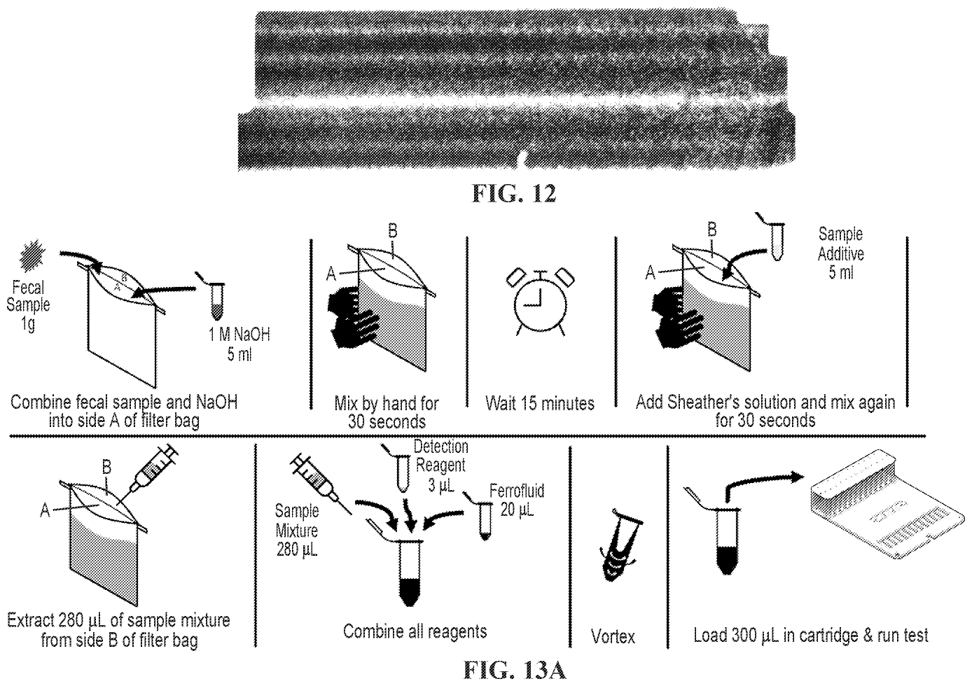

WGA has previously been described to bind to another apicomplexan oocyst, those of Toxoplasma gondii (Harito et al. 2017). Bushkin et al. 2013 also described fluorescence of Eimeria oocysts treated with fluorescently-labeled Dectin-1, while ConA was suggested as possibly binding to oocysts in previous publications (Fuller and McDougald 2002; Gavriilidou 2018) AlexaFluor-488 or CF-488 labeled lectins were tested for ability to stain oocysts in fecal samples. With respect to ConA, WGA, SBA, PNA, and MPL all bound and labeled the oocysts, MPL appears to have the greatest uniformity in binding to and fluorescently labeling Eimeria oocysts (see e.g., FIG. 7). ConA appears to have the greatest affinity for binding/capturing oocysts in place (see, e.g., FIGS. 8-9). Lectin affinity for the oocysts increased with NaOH pre-treatment of the oocysts, likely due to exposed glucan fibrils from the pretreatment, as evidenced by both increased capture and more uniform labeling. FIG. 7 showing a fecal sample fluorescently labeled with MPL with conjugated AlexaFfluor488 (image acquired on PIPER™, in the presence of ferrofluid).

FIG. 8 shows a ConA-coated cartridge showing bound oocysts fluorescently labeled with SYBR. The blue squares loosely mark the coated window, where the ConA is coated onto the cartridge and where oocysts are bound.

FIGS. 9a-b are images of the same corner of the ConA coated cartridge images shown in FIG. 8. FIG. 8, left, which is a 100 ms exposures of bound SYBR-stained oocysts. FIG. 8, right shows the same region with a 300 ms exposure after a 5 minute flush of the cartridge, showing oocysts still bound in place.

Divalent cations. Lectin binding can be potentiated by adding divalent cations to the material that should be bound. Ca2+ and Mn2+ may have potential for increasing the binding of lectins to their target, specifically that article looked at potentiating ConA binding to enteric bacteria (Porter et al. 1998). Accordingly, an experiment assessed if adding 0.5, 1.5 and 15 mM Ca2+ and Mn2+ to a sample increased oocyst binding to ConA, PNA, and SBA. The addition of Mn2+ qualitatively increased oocysts binding to the lectins.

Acid-Fast Stains. Auramine O is a weakly fluorescing, aniline dye that specifically binds to certain proteins, and has been described specifically for fluorescently staining coccidia (Bushkin et al. 2013). However, it is acutely toxic, an irritant, a health hazard, and an environmental hazard. Accordingly, Auramine O fluorescently labeled purified oocysts in Coccivac-B52 and oocysts from feces with and without pretreatment with NaOH and in the presence of ferrofluid. The resulting stained oocysts are generally very bright, and both internal and external structures are clear, some oocysts become extremely bright. FIG. 10 illustrates Auramine O fluorescently labeled oocysts in the presence of ferrofluid.

Mitochondrial Stains. MitoTracker™ Green and Mitoview™ Green are two examples of mitochondrial stains. MitoTracker Green purportedly localizes in mitochondria regardless of mitochondrial membrane potential, and the dye will stain live cells, although it is not well-retained after aldehyde fixation. MitoView dyes are fluorogenic mitochondrial stains for live cells. Accordingly, these dyes rapidly accumulate in mitochondria and can be imaged without washing. Both mitochondrial stains labeled oocysts fluorescently. FIG. 11 illustrates oocysts fluorescently labeled with MitoTracker Green.

In some embodiments, Eimeria can also be processed without NaOH and stained with any of the fluorescent labeled methods described above. When using SYBR in buffers without NaOH, lower concentrations of SYBR are used (in some embodiments) as the fluorescent signal is much brighter. (shown in FIG. 12)

FIG. 12 illustrates a sample labeled with SYBR without NAOH present (e.g., see above) in a lane (including an imaging window) of a processing assay (e.g., cartridge).

### EXAMPLE 3

Coccidiosis represents a significant determinant in the economic performance of poultry operations (Williams, 1999, Int. J. Parasitol. 29: 1209-1229; Chapman et al., 2013, Adv. Parasitol. 83: 93-171). Infections with protozoan parasites of the genus Eimeria cause coccidiosis in poultry, with E. acervuline, E. Brunetti, E. hagani, E. maxima, E. mites, E. mivati, E. necatrix, E. praecox, and E. tenella causing disease in chickens (McDougald, 2013, Diseases of Poultry, edited by Swayne et al., Wiley-Blackwell, 2013, pp. 1148-1166). Clinical and sub-clinical coccidiosis can cause increased feed conversion ratios (FCRs), poor uniformity within a flock, co-morbidities with increased instances of necrotic enteritis (NE) or salmonellosis, and increased mortality (Baba et al., 1982, Res. Vet. Sci. 33: 95-98; Williams, 2005, Avian Pathol. 34: 159-180; McDougald, 2013). Recent estimates of the global economic costs of coccidiosis put the figure at greater than 10 billion US dollars annually (Williams, 1999; Dalloul and Lillehoj, 2006, Expert Rev. Vaccines. 5: 143-163; Kadykalo et al., 2018, In J. Antimicrob Agents 51: 304-410; Blake et al., 2020, Vet. Res. 51: 115.).

Conventional disease control has included chemical treatments, ionophores, and vaccines (Peek and Landman, 2011, Vet. Q. 31: 143-161; Chapman, 2014, Poult. Sci. 93: 501-511). The emergence of resistant populations and the relatively uneven or poor performance of anticoccidial drugs has led to the frequent use of mixed products, combining two synthetic compounds or a synthetic compound and an ionophore (Peek and Landman, 2011; Chapman, 2014). The increasing pressure on food service companies to only buy poultry raised without any products classified as antibiotics, which includes ionophores in the United States (Chapman et al., 2010, Poult. Sci. 89: 1788-1801), the passage of the Veterinary Feed Directive (2015), and emphases on animal welfare (Thaxton et al., 2016, Poult. Sci. 95: 2198-2207.), in combination with the increasing prevalence of anticoccidial-resistant populations have prompted a need for increased understanding of interventional efficacy and alternate methods of coccidia control (Peek and Landman, 2011). Monitoring the efficacy of interventions has economic implications for the poultry producers and health implications for the birds (Jenkins et al., 2017, Avian Dis. 61: 214-220; Snyder et al., 2020, Poult. Sci. 100: 110-118).

Oocysts in feces or litter to understand field efficacy of interventional methods. using either the hemocytometer or McMaster method (Holdsworth et al., 2004, Vet. Parasitol. 121: 189-212). Diagnosis of coccidia on poultry farms historically and currently largely involves collecting fresh feces from the enclosure floor and then sending the sample to a laboratory for enumeration (oocysts per gram, or OPG). Both methods require personnel trained to identify, count, and report the number of oocysts, in addition to microscopes and other laboratory equipment. This manual microscopy process is time and labor intensive, and it takes significant practice before anew user is able to count oocysts consistently and achieve results that correlate with other users. As a result, OPG counts have been historically underutilized as a broad diagnostic tool (Ricciardi and Ndao, 2015, J. Biomol. Screen. 20: 6-21; Intra et al., 2016, Clin. Microbiol. Infect. 22: 279-284).

In addition to quantification of infection, identification of Eimeria species is needed for evaluation and control of the disease, as each species may respond differently to management strategies (McDougald et al., 1986, Avian Dis. 30: 690-4; Lee et al., 2010. J. Vet. Med. Sci. 72: 985-989). Conventionally, identification of Eimeria species is based on morphological features of the sporulated oocyst, sporulation time, and location/scoring of pathological lesions in the intestine, but the procedures require specialized expertise and are subjective (Long and Joyner, 1984, J. Protozool. 31: 535-541). Molecular methods are currently available to identify the Eimeria species (Jenkins et al., 2006, Avian Dis. 50: 632-635; Morris and Gasser, 2006, Biotechnol. Adv. 24: 590-603; Haug et al., 2007, Vet. Parasitol. 146: 35-45; Blake et al. 2008, Avian Pathol. 37: 89-94; Cantacessi et al., 2008, Vet. Parasitol. 154: 226-234; Vrba et al., 2010, Vet. Parasitol. 174: 183-190; Kumar et al., 2014, Vet. Parasitol. 199: 24-31; Blake et al. 2015, Proc. Natl. Acad. Sci. 112: E5343-E5350; Kawahara et al. 2008, Avian Dis. 52: 652-656; Barkway et al., 2011, BMC Vet. Res. 7: 67; Lalonde and Gajadhar, 2011, J Parasitol. 97: 725-730; Nolan et al., 2015, Parasitol. Int. 64: 464-470), but the lack of a rapid, low-cost test prevents their broad scale use. Additionally, one study suggested that region-specific differences in DNA sequences of Eimeria species may affect the accuracy of molecular detection methods (Loo et al., 2019, Sains Malaysiana. 48: 1425-1432).

To address the limitations of existing methods to measure oocysts in feces (low throughput, labor intensive), a novel method was developed for the automated identification and enumeration of oocysts from poultry fecal samples using the PIPER™ instrument (see above), which exploits ferrofluid-based cytometry to concentrate, manipulate, and count cells within a disposable microfluidic device. The assay enables a single technician to process up to 196 samples in a single work shift and eliminates the extensive training and subjectivity of manual microscopy methods by employing automated image analysis. This study introduces the PIPER™ coccidia diagnostic assay and compares the accuracy and variability of the novel diagnostic to a conventional hemocytometer counting method. It also correlates the size-based separation of oocyst counts with known individual species of oocysts.

Fecal samples were provided as blinded samples from commercial broiler producers throughout the United States. Additional purified oocyst samples were provided from Merck Animal Health (MAH, Madison, NJ) in the form of individual species samples of E. maxima, E. tenella, and E. acervulina, confirmed by oocyst morphology, lesion location in infected birds, and PCR testing. Southern Poultry Research, Inc. (Athens, GA) also provided purified and concentrated samples of E. acervulina, E. tenella, and E. maxima oocysts. All samples were shipped (wet ice) and stored at 4° C. Sample preparation is shown in FIGS. 13A and 13B. One gram of homogenized feces or sample to be analyzed (or 100 μL. of cleaned, single species) was measured into a 7 oz Whirl-Pak filter bag (B01385WA, Nasco, Fort Atkinson, WI). Five mL of 1 M NaOH (SK-80044-64, Cole-Palmer, Vernon Hills, IL) was added to the bag. The sample and NaOH were massaged by hand for 1 minute to create a uniform slurry. The sample was allowed to sit for 15 minutes and then Sheather's solution was added to a final concentration of 50% to prevent oocyst settling, and the sample was mixed again. A 280 μl aliquot of the slurry was removed from the filtered side of the bag to avoid any solids that could clog the microfluidic device and transferred to a new tube. The sample was mixed with 20 μl of ferrofluid and 3 μl of SYBR Green stain, vortexed to mix, and loaded into a single well of a PIPER™ cartridge.

Hemocytometer. A Neubauer hemocytometer was used to count oocysts as the reference method as previously described (Conway and McKenzie. Poultry Coccidiosis: Diagnostic and Testing Procedures (Third Edition). Blackwell Publishing Professional, 2007). Ten microliters of the prepared sample slurry (above) were loaded into the counting chamber. The oocysts in the four corner and center square of the etched slide (the quincunx) were counted to generate the hemocytometer count. The total number of oocysts counted was multiplied by the dilution and divided by the approximate volume of the area counted to report OPG. By this preparation, one oocyst counted in a chamber represented 20,000 OPG. Additional hemocytometer chambers were loaded with the same initial slurry and counted to generate sample replicate counts.

$$\frac{1 oocyst}{0.5 μL solution} * \frac{1 , 000 μL}{1 mL} * \frac{10 mL solution}{1 g feces} = 20 , 000 oocysts per gram ( OPG ) feces$$

PIPER™ instrument. Once the sample was prepared as above, 280 μL of the homogenized sample was added to a 2 mL microcentrifuge tube. 3 μL of a nucleic acid intercalating dye (Detection Reagent, P/N ANC-EIM001, Ancera LLC, Branford, CT) and 20 μL of Ferrofluid (P/N ANC-EIM001, Ancera LLC, Branford, CT) were added to the tube containing 280 μL of the homogenized sample, which was then mixed via vortex to create the assay mixture. During the incubation step of the sample preparation described above, the PIPER™ instrument was initialized, and a disposable cartridge (e.g., MagDrive™ cartridge, as part of the Coccidia Assay Kit, Ancera LLC, Branford, CT) was loaded on the prepared machine. The assay mixture was loaded into 1 of the 12 lanes in the disposable MagDrive™ Cartridge. Each lane is an independent, simultaneous test such that the PIPER™ is capable of running 12 unique or replicate (or some combination thereof) coccidia samples at the same time. After loading the cartridge, the user started the assay run using the PIPER's user interface. The PIPER's peristaltic pumping system uses pressure gradients to flow the assay mixture through the cartridge's channels, without the instrument contacting the assay fluids. Therefore, cross-contamination between cartridge lanes and previous/subsequent runs is prevented without the need to flush the instrument between runs. The PIPER™ generates a magnetic field using a printed circuit board (PCB) under the cartridge to push targets suspended within the ferrofluid mixture to the top of the cartridge (FIG. 13B) while the sample is flowing within the cartridge channel. The flow is stopped once the assay mixture has sufficiently filled the channel and imaging window. While the flow is stopped, the targets are immobilized at the top of the cartridge purely by the action of ferrofluid and magnetic force (Kose and Koser, 2012; Lab Chip 12: 190-196), and a built-in fluorescent microscope above the cartridge enables single-oocyst resolution.

Image recognition algorithm. To automate the PIPER™ analysis, an image recognition algorithm was developed to identify and enumerate oocysts in the acquired images. The image recognition algorithm was developed using a two-step deep learning approach that first runs a U-NET based segmentation model followed by a shallow Convolutional Neural Networks (CNN) classification model. Useful metadata metrics such as size and intensity are available for every target identified. This additional data is used to categorize the oocysts into large, medium, and small sizes based on their major and minor axes lengths. For this analysis, large oocysts were characterized as having major axis length greater than 27 microns, medium oocysts were categorized as having major axis length less than 27 microns and minor axis length greater than 18 microns, and small oocysts were characterized as having major axis length less than 27 microns and minor axis length less than 18 microns. For manual counting to assess the accuracy of the algorithm, trained analysts reviewed and counted oocysts in 67 PIPER™ images using the ImageJ software (U. S. National Institutes of Health, Bethesda, Maryland, USA, https://imagej.nih.gov/ij/).

Calibration to OPG. A total of 77 unique samples were assessed using both hemocytometer and PIPER™ to calibrate the PIPER™ counts to OPG. Each sample had 4 individual hemocytometer counts performed and most had 4 individual PIPER™ counts (six samples had 12 PIPER™ measurements; four samples had 3 PIPER™ measurements). The mean and standard deviation for each method was calculated per sample per method and used to determine a coefficient of variance (CV). Both mean and randomly paired individual PIPER™ and hemocytometer counts were graphed and evaluated to establish the calibration conversion from PIPER™ oocyst count to OPG. The samples had OPG levels ranging from 5000 to 5 million OPG by hemocytometer.

Linearity. Four samples of purified oocysts were prepared as described for cleaned, single species sample preparation above, with the modification that three preparations were made of each sample using different sampling volumes (100 μL, 30 μL, and 10 μL) in 5 mL of 1M NaOH. Each pseudo-dilution of sample was loaded on PIPER™ in quadruplicate (4 individual lanes) and used to assess the accuracy of counts below the limit of detection of the hemocytometer. Predicted counts were generated by multiplying the average 100 82 L count for each sample by either 0.3 or 0.1, respectively, and compared to the actual counts as determined by PIPER™.

Morphometrics and classification. Individual metrics on each identified oocyst were collected by the PIPER™ instrument and used to classify the oocysts as small, medium, or large using the criteria defined for the algorithm above. 100 μl samples of individual species of oocysts (E. acervuline, E. maxima, E. tenella) were prepared as described above and assessed by the PIPER™. The individual species samples utilized were cleaned oocyst samples prepared by MAH, with species identity confirmed by PCR.

Confirmatory testing. Paired PIPER™ and hemocytometer counts were obtained for an additional 96 field samples across three independent testing laboratories. The relative percent difference between the counting methods was computed using the formula:

$$RPD = \frac{average hemocytometer OPG - average PIPER OPG}{[ \frac{average hemocytometer OPG + average PIPER OPG}{2} ]}$$

Analysis. Graphics and data analyses were performed using R. To generate the calibration equation of PIPER™ counts to OPG, a linear regression was performed on the plotted points from paired hemocytometer and PIPER™ counts to generate a line with slope of approximately 425 and a calculated coefficient of determination (R-squared) of 0.979809 (FIG. 16). The linear fit was performed with and without forcing the y-intercept to go through zero and provided similar results. For confirmatory testing, the 95% confidence interval for the relative percent difference (RPD) lies within the interval [−2/3, 2/3] and was computed as the mean +/−two standard deviations of the RPD.

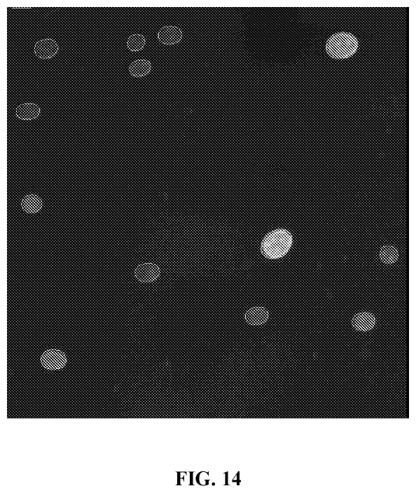

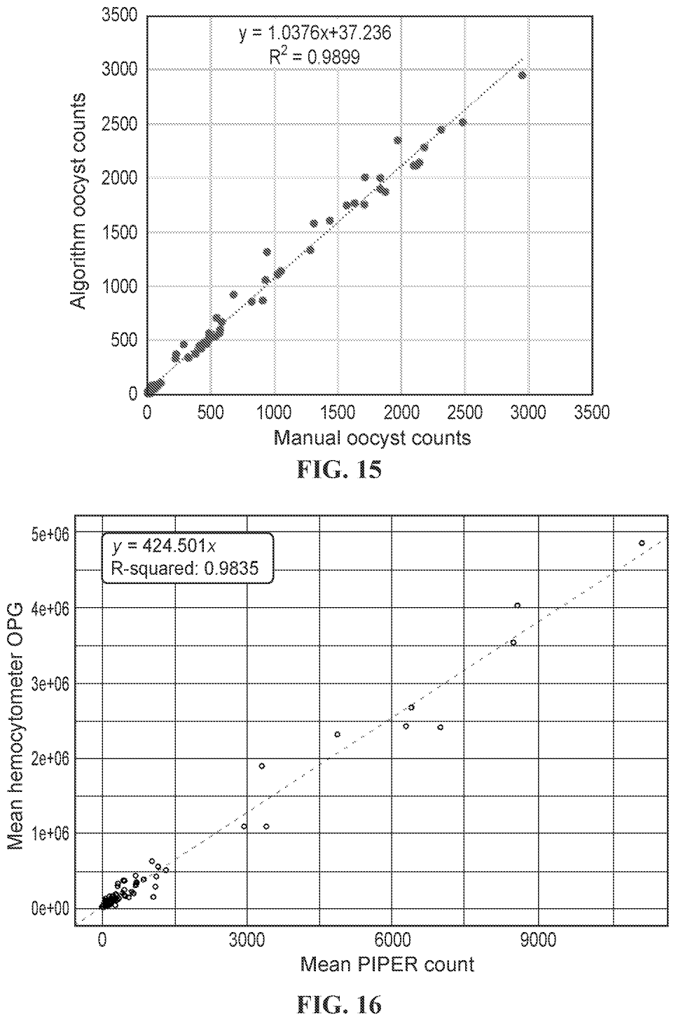

PIPER™ counting algorithm performance. The coccidia detection and quantification assay on the PIPER™ platform (FIGS. 13A and 13B) eliminates the subjective, labor intensive, manual microscopy steps that are associated with standard oocyst counting techniques. This is driven by the integrated deep learning oocyst detection algorithm that processes the scanned images. An exemplary image is shown in FIG. 14. The image in FIG. 14 was magnified 100%, detected oocysts are indicated by circles. Color discriminates oocysts is based on size: large (yellow), medium (blue), or small (green). To assess performance of the image recognition algorithm, more than 60 images generated by the coccidia detection assay on PIPER™ were manually reviewed by a technician trained to identify oocysts and simultaneously processed through the image recognition algorithm. A plot of manual counts against algorithm counts shows a slope near 1 and r² value of 0.99 (FIG. 15), suggesting algorithmic performance consistent with human oocyst identification.

Calibration of PIPER™ automated oocyst enumeration assay with hemocytometer counting method. The PIPER™ MagDrive™ technology enables concentration of oocysts in the imaging window as the sample flows through the cartridge (Kose and Koser, 2012). To calibrate the oocyst counts obtained by the PIPER™ assay with the conventional hemocytometer method of determining oocysts per gram (OPG), paired hemocytometer and PIPER™ data was collected for 77 unique samples prepared by the PIPER™ sample preparation method. Counts obtained by the two methods were plotted against each other (FIG. 16), and the linear fit of hemocytometer counts in OPG to PIPER™ total oocyst counts was used to generate a calibration equation to convert PIPER™ counts to OPG. Based on this equation, one oocyst count on PIPER™ corresponds to an oocyst concentration in the sample of 425 OPG (FIG. 16). Based on this calibration, fewer measurements are needed to detect low OPG levels by the PIPER™ method, where one oocyst in the imaging window corresponds to 425 OPG, compared to the hemocytometer method, in which a single oocyst detected in 1 quincunx of a hemocytometer chamber is equivalent to 20,000 OPG.

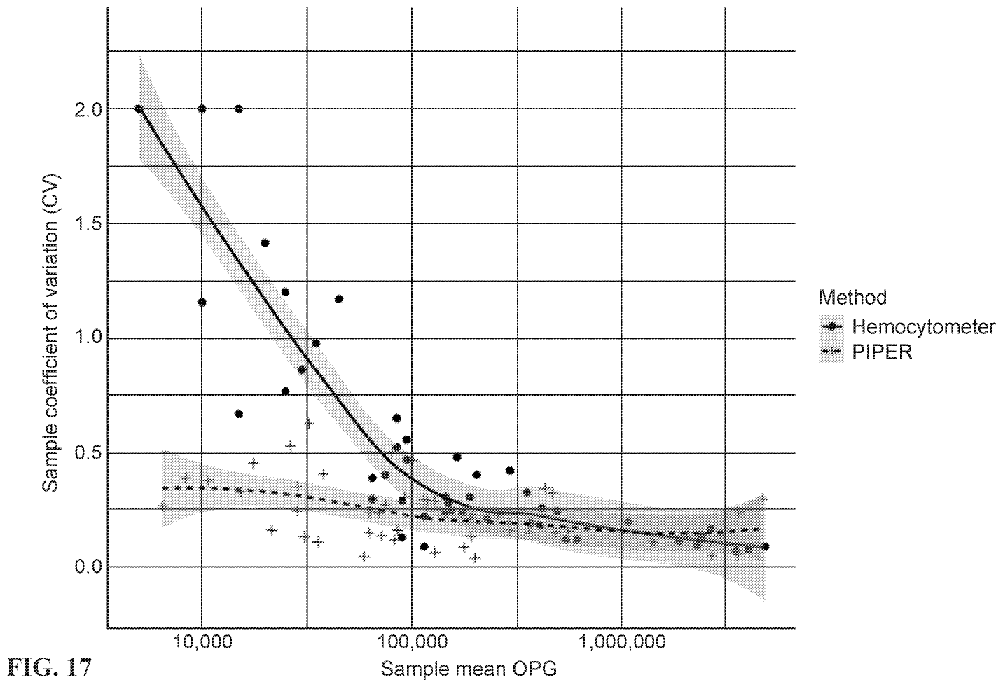

Repeatability of PIPER™ automated oocyst enumeration. The coefficient of variation (CV) was calculated for each unique sample (n=77) from the calibration study, and the CVs of hemocytometer and PIPER™ counts are graphically represented in FIG. 17. CVs for the PIPER™ assay were consistent across a 3-log range of OPG and were lower than hemocytometer CVs at concentrations below 100,000 OPG (FIG. 17). This data suggests that the PIPER™ assay has the potential to be more reliable than the hemocytometer counting method across a wider range of OPG levels.

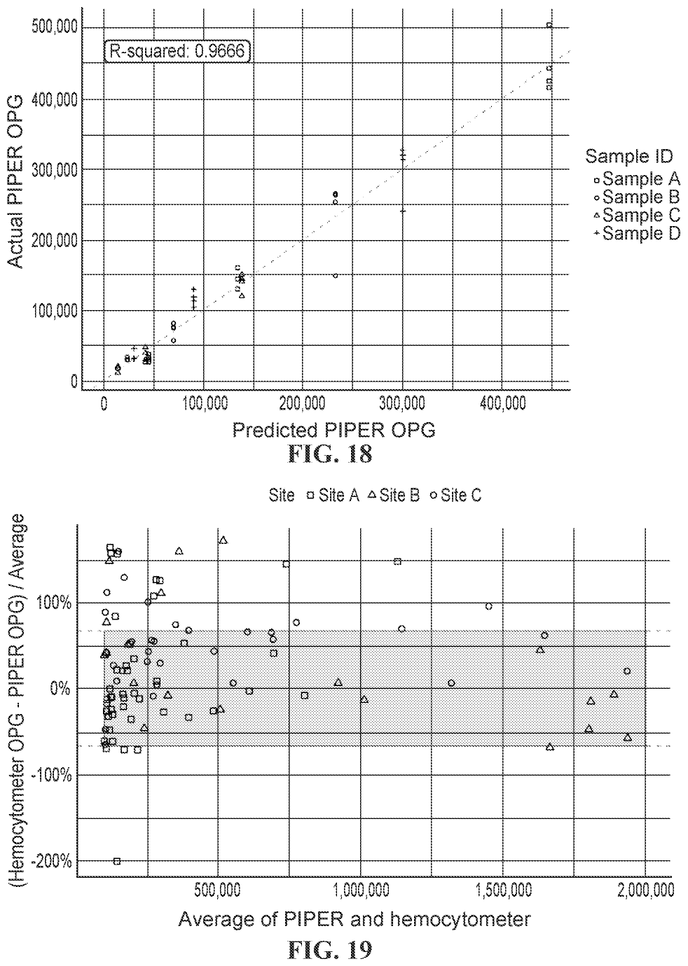

PIPER™ assay linearity and performance at low concentrations. The comparison of CVs showed that hemocytometer OPG counts were increasingly variable at concentrations less than 100,000 OPG, even with the same technicians. This is consistent with the reported lower limit for accurate counting of cells via hemocytometer. The linearity of the assay across three different volumes of each of four, cleaned, oocyst samples was evaluated to assess a predicted linear relationship of sample concentrations below the levels at which the hemocytometer could be considered reliable. The average total counts on PIPER™ for four replicates of each sample at each volume were determined. The average count for the 1× volume of each sample was multiplied by 0.3 or 0.1 to calculate a predicted count for the smaller volumes of the same sample. These predicted counts were plotted against the actual counts for all samples and a linear regression performed to assess the linearity of the assay below the lower limit of accuracy or repeatability for the hemocytometer method. This analysis showed a linear relationship between the actual and predicted values with an r² of 0.97 (FIG. 18).

Morphometrics and classification. Metadata such as major axis, minor axis, area, and intensity are documented for every oocyst identified by the image recognition algorithm. These data are used by the algorithm to classify oocysts as small, medium, or large (FIG. 14). To evaluate the differences in oocyst sizes detected by the algorithm for different Eimeria species, samples of cleaned, individual Eimeria species were prepared following the Coccidia Detection and Enumeration Assay on PIPER™. The study included 12 lane replicates each of E. acervulina, E. tenella, and E. maxima. In this study, 76% of E. acervulina oocysts were classified as small oocysts, 81% of E. tenella were classified as medium oocysts, and 96% of E. maxima were classified as large oocysts (Table 1). This data is consistent with previous morphometric analysis of these species (Haug et al., 2008, Avian Pathol. 37: 161-170) and suggests that the ability of the image recognition algorithm to bin oocyst counts into size categories can help provide additional information about the population of coccidia that is present in the sample.

TABLE 1

| | Total | Small | Medium | Large | % Small | % Medium | % Large |
|---|---|---|---|---|---|---|---|
| | oocyst | oocyst | oocyst | oocyst | oocyst | oocyst | oocyst |
| Sample | counts | counts | counts | counts | counts | counts | counts |
| E. acervulina | 13141 | 9993 | 3148 | 0 | 76% | 24% | 0% |
| E. maxima | 3791 | 40 | 117 | 3634 | 1% | 3% | 96% |
| E. tenella | 8503 | 1511 | 6862 | 130 | 18% | 81% | 1% |

Confirmatory testing. To assess the accuracy of the PIPER™ calibration to OPG, an additional 96 field samples with hemocytometer OPG counts in the range of 100,000 to 2 million were evaluated across three independent laboratories. All samples included paired PIPER™ and hemocytometer counts. Agreement between the two methods was assessed by plotting the relative percent difference between methods against the average OPG of the two methods. Based on this analysis, 75% of the samples fell within a 2-fold difference or less (FIG. 19). Taken together, these data suggest that the PIPER™ assay could provide comparable performance to the hemocytometer method with improved throughput and ease of use, enabling monitoring of oocyst cycling in flocks to understand trends and efficacy of interventions that is not practical currently.

### Discussion

The use of OPG to quantify Eimeria levels in a flock has been used to categorize flocks at risk of decreased performance levels (Haug et al., 2008). Recent data suggests that OPG measurements could provide insight in evaluating efficacy and the performance of intervention strategies, potentially linking performance metrics such as average daily gain or feed conversion ratios (Chasser et al., 2020; Poult. Sci. 99: 886-892) and providing guidance for veterinarians and producers on the timing and appropriateness of adding different anticoccidial compounds, nutraceuticals, or vaccination protocols (Jenkins et al., 2017). Despite the need for quantitative tools that can rapidly and accurately determine parasite numbers to guide interventions and disease control, classical microscopy methods to identify Eimeria species are time consuming, can be subjective, and are difficult to scale up for medium to high-throughput applications (Joyner and Long, 1974, Avian Pathol. 3: 145-157; Long and Joyner, 1984). The PIPER™ assay described in this example enables the automated identification and quantification of oocysts from fecal samples. Unlike traditional hemocytometer and McMaster methods which require sample processing in batch, one PIPER™ cartridge can support the analysis of twelve fecal samples in parallel on one instrument. Sample preparation is simple, and run time on the instrument is less than an hour. This method eliminates the extensive training and subjectivity of manual microscopy methods and increases throughput of obtaining OPG measurements to up to 192 per technician in a single work shift with 2 PIPER™ instruments. Increased scalability can conceivably be achieved with incorporation of robotics and additional PIPER™ instruments. Since the PIPER™ assay can generate large amounts of data on oocyst populations in a short timeframe, this method could enable veterinarians to assess coccidia load at a populational level by surveying fecal samples from individual birds, providing more granularity in understanding the efficacy of coccidia interventions than possible by necropsy alone. Currently, necropsies are done on a couple of birds at approximately 9 weeks, providing information on health of the flock at the end of life. With the PIPER™ assay, OPG monitoring can be done on the entire population throughout the life cycle, enabling real-time monitoring of treatment efficacy. Furthermore, since the PIPER™ assay can be performed directly on fecal samples, monitoring does not require sacrificing the birds.

Other technologies, such as flow cytometry, can be used to sort and enumerate cells based on fluorescent signals. However, flow cytometry is prone to clogging, requires instrument-specific calibration, and cannot be used with complex environmental samples like feces without extensive sample preparation. The PIPER™ MagDrive™ technology uses a uniquely patterned printed circuit board to generate magnetic force on a ferrofluid composed of superparamagnetic nanoparticles for flow-based manipulation of cells. It allows for efficient detection of oocysts and enumeration from complex field samples, including feces and intestinal contents, using the PIPER™ technology, and the results of confirmatory testing at field sites showed a strong correlation with the classical hemocytometer method. Furthermore, CVs for the PIPER™ assay were consistent across a 3-log range of OPG and were lower than hemocytometer CVs at concentrations below 100,000 OPG, suggesting better reliability of the PIPER™ assay across a wider dynamic range. Based on the calibration of PIPER™ counts to hemocytometer counts, a single oocyst count observed on PIPER™ translates to approximately 425 OPG, which is significantly below the limit of detection of a single hemocytometer chamber (1 oocyst=20,000 OPG), suggesting that this system requires fewer measurements to detect low oocyst levels. Further sensitivity could conceivably be achieved through a sample concentration step using centrifugation or filtration.

### REFERENCES

Armitage B. A. (2005) Cyanine Dye—DNA Interactions: Intercalation, Groove Binding, and Aggregation. In: Waring M. J., Chaires J. B. (eds) DNA Binders and Related Subjects. Topics in Current Chemistry, vol 253. Springer, Berlin, Heidelberg.

Bushkin G G, Motari E, Carpentieri A, Dubey J P, Costello C E, Robbins P W, Samuelson J. 2013. Evidence for a structural role for acid-fast lipids in oocyst walls of Cryptosporidium, Toxoplasma, and Eimeria. mBio 4(5):e00387-13. Doi:10.1128/mBio.00387-13.

Harito J B, Campbell A T, Tysnes K R, Dubey J P, Robertson L J, 2017. Lectin-magnetic separation (LMS) for isolation of Toxoplasma gondii oocysts from concentrated water samples prior to detection by microscopy or qPCR. Water Res. 2017; 114:228-236. doi:10.1016/j.watres.2017.02.044

Tanriverdi S, Tanyeli A, Başlamişli F, Köksal F, Kilinç Y, Feng X, Batzer G, Tzipori S, Widmer G. Detection and genotyping of oocysts of Cryptosporidium parvum by real-time PCR and melting curve analysis. J Clin Microbiol. 2002 September; 40(9):3237-44. doi: 10.1128/jcm.40.9.3237-3244.2002. PMID: 12202559; PMCID: PMC130769. Baba, E., T. Fukata, and A. Arakawa. 1982. Establishment and persistence of Salmonella typhimurium infection stimulated by Eimeria tenella in chickens. Res. Vet. Sci. 33: 95-98.

Barkway, C. P., R. L. Pocock, V. Vrba, and D. P. Blake. 2011. Loop-mediated isothermal amplification (LAMP) assays for the species-specific detection of Eimeria that infect chickens. BMC Vet. Res. 7: 67.

Blake, D. P., Z. Qin, J. Cai, and A. L. Smith. 2008. Development and validation of real-time polymerase chain reaction assays specific to four species of Eimeria. Avian Pathol. 37: 89-94.

Blake, D. P., E. L. Clark, S. E. MacDonald, V. Thenmozhi, K. Kundu, R. Garg, I. D. Jatau, S. Ayoade, F. Kawahara, A. Moftah, and A. J. Reid. 2015. Population, genetic, and antigenic diversity of the apicomplexan Eimeria tenella and their relevance to vaccine development. Proc. Natl. Acad. Sci. 112: E5343-E5350.

Blake, D. P., J. Knox, B. Dehaeck, B. Huntington, T. Rathinam, V. Ravipati, S. Ayoade, W. Gilbert, A. O. Adebambo, I. D. Jatau, M. Raman, D. Parker, J. Rushton, and F. M. Tomley. 2020. Re-calculating the cost of coccidiosis in chickens. Vet. Res. 51: 115.

Bushkin, G. G., E. Motari, A. Carpentieri, J. P. Dubey, C. E. Costello, P. W. Robbins, and J. Samuelson. 2013. Evidence for a structural role for acid-fast lipids in oocyst walls of Cryptosporidium, Toxoplasma, and Eimeria. mBio. 4: e00387-13.

Cantacessi, C., S. Riddell, G. M. Morris, T. Doran, W. G. Woods, D. Otranto, and R. B. Gassera. 2008. Genetic characterization of three unique operational taxonomic units of Eimeria from chickens in Australia based on nuclear spacer ribosomal DNA. Vet. Parasitol. 154: 226-234.

Chapman, H. D., T. K. Jeffers, and R. B. Williams. 2010. Forty years of monensin for the control for the control of coccidiosis in poultry. Poult. Sci. 89: 1788-1801.

Chapman, H. D., J. R. Barta, D. Blake, A. Gruber, M. Jenkins, N. C. Smith, X. Suo, and F. M. Tomley. 2013. A selective review of advances in coccidiosis research. Adv. Parasitol. 83: 93-171. Chapman, H. D. 2014. Milestones in avian coccidiosis research: a review. Poult. Sci. 93: 501-511.

Chasser, K. M., A. F. Duff, K. M. Wilson, W. N. Briggs, J. D. Latorre, J. R. Barta, and L. R. Bielke. 2020. Evaluating fecal shedding of oocysts in relation to body weight gain and lesion scores during Eimeria infection. Poult. Sci. 99: 886-892.

Conway, D. P., and M. E. McKenzie. Poultry Coccidiosis: Diagnostic and Testing Procedures (Third Edition). Blackwell Publishing Professional, 2007.

Dalloul, R. A., and H. S. Lillehoj. 2006. Poultry coccidiosis: recent advancements in control measures and vaccine development. Expert Rev. Vaccines. 5: 143-163.

Haug, A., P. Thebo, and J. G. Mattsson. 2007. A simplified protocol for molecular identification of Eimeria species in field samples. Vet. Parasitol. 146: 35-45.

Haug, A., A.-G. Gjevre, P. Thebo, J. G. Mattsson, and M. Kaldhusdal. 2008. Coccidial infections in commercial broilers: epidemiological aspects and comparison of Eimeria species identification by morphometric and polymerase chain reaction techniques. Avian Pathol. 37: 161-170.

Holdsworth, P. A., D. P. Conway, M. E. McKenzie, A. D. Dayton, H. D. Chapman, G. F. Mathis, J. T. Skinner, H. C. Mundt, R. B. Williams, and World Association for the Advancement of Veterinary Pathology. 2004. World Association for the Advancement of Veterinary Parasitology (WAAVP) guidelines for evaluating the efficacy of anticoccidial drugs in chickens and turkeys. Vet. Parasitol. 121: 189-212.

Intra, J., E. Tavema, M. R. Sala, R. Falbo, F. Cappellini, and P. Brambilla. 2016. Detection of intestinal parasites by use of the cuvette-based automated microscopy analyzer sediMAX. Clin. Microbiol. Infect. 22: 279-284.

Jenkins, M. C., K. Miska, and S. Klopp. 2006. Improved polymerase chain reaction technique for determining the species composition of Eimeria in poultry litter. Avian Dis. 50: 632-635.

Jenkins, M. C., C. Parker, and D. Ritter. 2017. Eimeria oocyst concentrations and species composition in litter from commercial broiler farms during anticoccidial drug or live Eimeria oocyst vaccine control programs. Avian Dis. 61: 214-220.

Joyner, L. P., and P. I. Long. 1974. The specific characters of the Eimeria, with special reference to the coccidia of the fowl. Avian Pathol. 3: 145-157.

Kadykalo, S., T. Roberts, M. Thompson, J. Wilson, M. Lang, and O. Espeisse. 2018. The value of anticoccidials for sustainable global poultry production. In J. Antimicrob Agents 51: 304-410.

Kawahara, F., K. Taira, S. Nagai, H. Onaga, M. Onuma, and T. Nunova. 2008. Detection of five avian Eimeria species by species-specific real-time polymerase chain reaction assay. Avian Dis. 52: 652-656.

Kose, A. R. and H. Koser. 2012. Ferrofluid mediated nanocytometry. Lab Chip 12: 190-196.

Kumar, S. R. Garg, A. Moftah, E. L. Clark, S. E. Macdonald, A. S. Chaudhry, O. Sparagano, P. S. Banerjee, K. Kundu, F. Tomley, and D. P. Blake. 2014. An optimised protocol for molecular identification of Eimeria from chickens. Vet. Parasitol. 199: 24-31.

Lalond, L. and A. A. Gajadhar. 2011. Detection and differentiation of coccidian oocysts by real-time PCR and melting curve analysis. J Parasitol. 97: 725-730.

Lee, B. H., W. H. Kim, J. Jeong, J. Yoo, Y. K. Kwon, B. Y. Jung, J. H. Kwon, H. S. Lillehoj, and W. Min. 2010. Prevalence and cross-immunity of Eimeria species on Korean chicken farms. J. Vet. Med. Sci. 72: 985-989.

Long, P. I. and L. P. Joyner. 1984. Problems in the identification of species of Eimerial. J. Protozool. 31: 535-541.

Loo, S.-S., L.-S. Lim, N. A. Efendi, D. P. Blake, S.-I. Kawazu, K.-L. Wan. 2019. Comparison of molecular methods for the detection of Eimeria in domestic chickens in Malaysia. Sains Malaysiana. 48: 1425-1432.

Morris, G. M. and R. B. Gasser. 2006. Biotechnological advances in the diagnosis of avian coccidiosis and the analysis of genetic variation in Eimeria. Biotechnol.Adv. 24: 590-603.

McDougald, L. R., L. Fuller, and J. Solis. 1986. Drug-sensitivity of 99 isolates of coccidia from broiler farms. Avian Dis. 30: 690-4.

McDougald, L. R., and S. H. Fitz-Coy. “Coccidiosis.” Diseases of Poultry, edited by D. E. Swayne, J. R. Glisson, L. R. McDougald, L. K. Nolan, D. L. Suarez, and V. L. Nair, Wiley-Blackwell, 2013, pp. 1148-1166.

Nolan, M. J., F. M. Tomley, P. Kaiser, and D. P. Blake. 2015. Quantitative real-time PCR (qPCR) for Eimeria tenella replication—Implications for experimental refinement and animal welfare. Parasitol. Int. 64: 464-470.

Peek, H. W., and W. J. M. Landman. 2011. Coccidiosis in poultry: anticoccidial products, vaccines and other prevention strategies. Vet. Q. 31: 143-161.

R Core Team. 2018. R: A language and environment for statistical computing. R Foundation for Statistical Computing, Vienna, Austria. URL https://www.R-project.org/

Ricciardi, A., and M. Ndao. 2015. Diagnosis of parasitic infections: what's going on? J. Biomol. Screen. 20: 6-21.

Snyder, R. P., M. T. Guerin, B. M. Hargis, G. Page, and J. R. Barta. 2021. Monitoring coccidia in commercial broiler chicken flocks in Ontario: comparing oocyst cycling patterns in flocks using anticoccidial medications or live vaccination. Poult. Sci. 100: 110-118.

Thaxton, Y. V., K. D. Christensen, J. A. Mench, E. R. Rumley, C. Daugherty, B. Feinberg, M. Parker, P. Siegel, and C. G. Scanes. 2016. Symposium: Animal welfare challenges for today and tomorrow. Poult. Sci. 95: 2198-2207.

Vrba, V., D. P. Blake, and M. Poplstein. 2010. Quantitative real-time PCR assays for detection and quantification of all seven Eimeria species that infect the chicken. Vet. Parasitol. 174: 183-190.

Williams, R. B. 1999. A compartmentalized model for the estimation of the cost of coccidiosis to the world's chicken production industry. Int. J. Parasitol. 29: 1209-1229.

Williams, R. B. 2005. Intercurrent coccidiosis and necrotic enteritis of chickens: rational, integrated disease management by maintenance of gut integrity. Avian Pathol. 34: 159-180.

What is currently claimed is:

1. An assay system configured to at least one of detect, enumerate, and characterize one or more parasite oocyst or egg from at least one fecal sample or a plurality of samples, comprising:
a ferrofluidic assay device configured to receive a ferrofluidic cartridge;
the ferrofluidic cartridge including a plurality of windows;
an imager configured to image each window of the cartridge either separately or together, wherein detecting, enumerating, and characterizing one or more parasite oocyst or egg is determined by an analysis of one or more images;
a controller configured to control at least one of the ferrofluidic assay device, the ferrofluidic cartridge, and the imager;
and
assay processing components comprising at least one of reagents, and controls
wherein the system is configured to at least one of: separate any and all of parasite oocysts or eggs; move or otherwise locate the parasite oocysts or eggs to one or more of the windows; detect, enumerate/count, and/or characterize the parasite oocysts or eggs.

2. The system of claims 1, wherein any parasite oocyst or egg contained within the plurality of samples are at least one of:
labeled and visualized by fluorescence, and
unlabeled and visualized by respective intrinsic auto-fluorescence.

3. The system of any of claims 1-2, wherein:
the samples are mixed with ferrofluid prior to being flowed past at least one window; and
the device establishes at least one electromagnetic field to act on the ferrofluid so as to direct at least one of parasite oocysts or eggs contained in the sample to a specific area or location.

4. The system of any of claims 1-4, wherein the ferrofluid includes at least one density modifying agents.

5. The system of claim 4, wherein the density modifying agent comprises at least one of a concentrated sugar solution, and Sheather's solution.

6. The system of any of claims 1-5, where each sample is at least one of processed and mixed with one or more of sodium hydroxide, a sodium hydroxide solution and/or detergents.

7. The system of claim 6, wherein the sodium hydroxide is mixed with each sample by mixing equal volumes of sample and sodium hydroxide solution, mixing a 1:2 volume:volume of sample and sodium hydroxide solution, mixing a 1:3 volume:volume of sample and sodium hydroxide solution, mixing a 1:4 volume:volume of sample and sodium hydroxide solution, mixing a 1:5 volume:volume of sample and sodium hydroxide solution, mixing a 2:1 volume:volume of sample and sodium hydroxide solution, mixing a 3:1 volume:volume of sample and sodium hydroxide solution, mixing a 4:1 volume:volume of sample and sodium hydroxide solution, or mixing a 5:1 volume:volume of sample and sodium hydroxide solution, and optionally incubating the mixture prior to flowing the mixture over a capture region of the system.

8. The system of claim 6 or 7, wherein the concentration of sodium hydroxide is: between 0.01 molar (M) and 2M, between 0.2M and 1.2M, or at about 1M.

9. The system of claim 7 or 8, wherein mixture is incubated for less than 45 minutes, between 1-45 minutes, less than 60 minutes, between 1-60 minutes, about 15 minutes, about 30 minutes, about 1 hour, about 2 hours, about 3 hours, about 4 hours, about 5 hours, about 6 hours, about 12 hours, or between 1-24 hours prior to the samples being analyzed.

10. The system of any of claims 3-9, wherein the samples are added to ferrofluid at a predetermined ratio, e.g., 1 part ferrofluid to 14 parts sample, 1 part ferrofluid to 10 parts sample, 1 part ferrofluid to 9 parts sample, 1 part ferrofluid to 8 parts sample, 1 part ferrofluid to 7 parts sample, 1 part ferrofluid to 6 parts sample, 1 part ferrofluid to 5 parts sample, 1 part ferrofluid to 4 parts sample, 1 part ferrofluid to 3 parts sample, 1 part ferrofluid to 2 parts sample, 1 part ferrofluid to 1 part sample, or 2 parts ferrofluid to 1 part sample.

11. The system of any of claims 1-10, further comprising at least one of a hemocytometer and a McMaster chamber.

12. The system of claim 11, where at least one of the hemocytometer and McMaster chamber are configured to count the number of parasite oocysts and/or eggs, characterize the parasite oocysts and/or eggs, and/or permit the fluorescence excitation/emission of labeled parasite oocysts and/or eggs in the samples using wavelengths specific to the fluorophore used.

13. The system of any of claims 1-12, wherein the system further comprises a capture region adjacent to each window, configured to capture one or more predetermined parasites and/or oocysts; wherein each capture region is coated with a binding agent configured to bind with surface features on at least one of the parasite oocysts and/or eggs.

14. The system of claim 12 or 13, wherein the binding agent comprises at least one of an antibody, aptamer, lectin, and mucin.

15. The system of any of claims 12-14, wherein the binding agent is bound to the capture region via at least one of avidin, streptavidin, neutravidin, and any other modified binder.

16. The system of any one of the preceding claims, wherein the system is configured to flow at least one of the plurality of samples into cartridge for a predetermined period of time.

17. The system of claim 16, wherein after the predetermined period of time, the system is configured to stop the flow of the at least one sample into the cartridge, and the captured parasite oocysts and/or eggs are counted.

18. The system of claim 17, wherein the system is additionally configured to characterize the counted parasite oocysts and/or eggs.

19. The system of any of claims 1-18, wherein each sample is exposed to at least one fluorophore or fluorophore labeled agent.

20. The system of claim 19, wherein exposing each sample to at least one fluorophore or fluorophore labeled agent is configured such that at least one of SYBR, fluorescent labeled lectins, fluorescent labeled antibodies, acid fast stains, membrane stains, and fluorophore labeled in-situ-hybridization probes can be visualized.

21. The system of any of claims 13-20, wherein parasite oocysts and/or eggs are labeled after capture in a capture region/zone.

22. The system of any claims 1-21 wherein parasite oocysts and/or eggs are pre-labeled prior to processing.

23. The system of any of claims 1-22, wherein the system is configured to characterize parasites by at least one of size, shape, and other morphological parameter.

24. The system of any of claims 1-23, wherein the system is further configured to characterize a level of sporulation of individual oocyst contained in at least one sample.

25. The system of any of claims 1-24, wherein the system is further configured to determine a state of oocyst sporulation.

26. The system of any of claims 1-25, wherein the system is further configured to visualize a level of sporulation of individual oocysts contained in at least one sample.

27. The system of claim 26, wherein sporulation is visualized by exposing the at least one sample with at least one staining agent, the at least one staining agent may comprise a DNA intercalating dye, and wherein the DNA intercalating dye can comprise SYBR.

28. The system of any of claims 1-27, wherein the oocysts and/or eggs are from the parasite Coccidia.

29. The system of claim 28, wherein the coccidia comprises at least one of genera Toxoplasma, Cryptosporidium, Cyclospora, Eimeria, and Cyclospora.

30. The system of claim 29, wherein the coccidia comprises Eimeria.

31. A method for detecting parasitic infection in a sample in an assay system comprising:
preparing a plurality of fecal samples for analysis, wherein preparing comprises, for each sample:
labeling at least each of a first tube and a second tube placing a predetermined amount of sample within the first tube;
placing a predetermined amount of NaOH at a pre-determined concentration in the first tube;
vortexing the first tube for a first predetermined period of time;
placing a predetermined amount of Sheather's sugar solution to the first tube;
vortexing the first tube for a second predetermined period of time;
optionally removing an amount of the sample from the first tube and determining a reference count of oocysts;
transferring a third predetermined amount of each fecal-NaOH mixture of each first tube into a respective second tube;
adding a fourth predetermined amount of ferrofluid and a fifth predetermined amount of SYBR into each respective second tube;
vortexing each respective second tube for a third predetermined amount of time
add the vortexed samples to lanes of an assay cartridge having a plurality of channels/lanes, each channel configured with at least one capture region/zone having at least one unique agent for binding with at least one specific parasite oocyst or egg;
flowing the mixture over at least one of the channels of the cartridge, wherein electromagnetic fields produced by the system and acting on the ferrofluid push up oocysts contained in the at least one sample to a capture region proximate a channel window provided for each channel;
scanning each window of each channel to produce a respective scan image thereof;
processing each respective scan image so as to at least one of identify and count each oocyst imaged from a respective channel window;
converting each count from each processed image to oocysts per gram of original fecal sample; and
optionally identifying and measuring the major axis of each counted oocyst such that the number of small (<18 μm), medium (18-25 μm), and large (>25 μm) oocysts are counted for each sample and optionally converted to oocysts per gram of original fecal sample.

32. The method of claim 31, wherein the sample is a fecal sample.

33. The method of claim 31 or 32, wherein the first tube is a vortexing tube, wherein the first tube is a 1 mL to 20 mL vortexing tube, a 5 mL to 10 mL vortexing tube, a 5 mL vortexing tube, or a 10 mL vortexing tube.

34. The method of any one of claim 31-33 wherein the second tube is a 1.7 mL to 2 mL vortexting tube.

35. The method of any one of claim 31-34, wherein the predetermined amount of sample is 0.1 g, 0.2 g, 0.3 g, 0.4 g, 0.5 g, 0.6 g, 0.7 g, 0.8 g, 0.9 g, 1 g, or is between 0.1 g and 2 g, between 0.1 g and 1 g, or between 0.2 g and 0.5 g.

36. The method of any one of claim 31-35, wherein the predetermined amount NaOH is 1 mL, 2 mL, 3 mL, 4 mL, 5 mL, or is between 1 mL and 5 mL.

37. The method of any one of claim 31-36, wherein the pre-determined concentration of NaOH is 0.1M, 0.2M, 0.3M, 0.4M, 0.5M, 0.6M, 0.7M, 0.8M, 0.9M, 1M, is between 0.2M and 1M, is between 0.1M and 1M.

38. The method of any one of claims 31-37, wherein the first predetermined period of time is 30 seconds, 45 seconds, or 60 seconds, or is 30 seconds to 60 seconds.

39. The method of any one of claims 31-38, wherein the predetermined amount of Sheather's sugar solution is 1 mL, 2 mL, 3 mL, 4 mL, 5 mL, or is between 1 mL and 5 mL.

40. The method of any one of claims 31-39, wherein the second predetermined period of time is 30 seconds, 45 seconds, or 60 seconds, or is from 30 seconds to 60 seconds.

41. The method of any one of claims 31-40, wherein the third predetermined amount is 200 μL, 210 μL, 220 μL, 230 μL 240 μL, 250 μL, 260 μL, 270 μL, 280 μL, 290 μL, 300 μL, between 200-300 μL, or between 260-300 μL.

42. The method of any one of claims 31-41, wherein the fourth predetermined amount is 10 μL 20 μL, 30 μL, 40 μL, 50 μL, 60 μL or between 10-60 μL of ferrofluid.

43. The method of any one of claims 31-42, wherein the fifth predetermined amount is 1 μL, 2μL, 3 μL, 4 μL, 5 μL or between 1-5 μL.

44. The method of any one of claims 31-43, wherein the third predetermined amount of time is 30 seconds, 45 seconds, or 60 seconds, or is from 30 seconds to 60 seconds.

45. The method of any one of claims 31-44, wherein 200 μL, 210 μL, 220 μL, 230 μL 240 μL, 250 μL, 260 μL, 270 μL, 280 μL, 290 μL, 300 μL, between 200-300 μL, or between 260-300 μL of the vortexed samples are added to lanes of the assay cartridge.

46. A method for detecting parasitic infection in fecal samples in an assay system comprising:
optionally placing a predetermined amount of animal fecal matter into a first side (“fecal side”) of at least a two-sided sample filter bag (“sample bag”);
adding a predetermined amount of NaOH to the first side of the sample bag forming a first mixture;
performing a first homogenization of the mixture, via, for example, massaging the mixture for a first predetermined period of time;
incubating the mixture for a second predetermined period of time;
adding in a predetermined amount of Sheather's solution to a second side of the sample bag (“filtered side”);
performing a second homogenization of the mixture, via, for example, massaging the mixture of a second predetermined period of time;
removing an aliquot of a predetermined amount from the filtered side of the bag;
transferring the aliquot to a tube of predetermined size;
adding a predetermined amount of ferrofluid to the tube;
vortexing the tube for a predetermined period of time;
transferring the contents of the tube to a single sample well of the assay cartridge;
flowing each sample from each sample well into an imaging window;
push all cells to the surface of the imaging zone;
optionally repeating the above-noted steps a plurality of times so as to utilize a corresponding number of sample wells, such that, each sample corresponds to a unique sample well,
optionally flowing each sample from each sample well into at least one imaging/capture zone of the assay cartridge so as to capture at least one specific parasite oocyst or egg contained in the sample;
acquiring image of each imaging zone;
determining a number of parasite oocysts or eggs present in the imaging zone; and
determining a size distribution of the parasite oocysts or eggs captured in the at least one imaging zone.

47. The method of claim 46, wherein the method further comprises adding in a predetermined amount a fluorophore or fluorophore labeled agent to the tube.

48. The method of claim 46 or 47, wherein the parasite ocysts and/or eggs are labeled with at least one fluorophore or fluorophore labeled agent, wherein the fluorophore or fluorophore labeled agent is selected from the group consisting of SYBR, fluorescent labeled lectins, fluorescent labeled antibodies, acid fast stains, membrane stains, or fluorophore labeled in-situ-hybridization probes.

49. The method of any one of claims 46-48, wherein the fluorophore or fluorophore labeled agent is added prior to mixing the sample with ferrofluid.

50. The method of claim 46 where no fluorescent labeling agent is used, and the parasite oocysts and/or eggs are monitored by their intrinsic fluorescence.

51. The method of claim 46-50 wherein the predetermined amount of animal fecal matter is between 0.1 g and 5 g.

52. The method of claim 46 or 51, wherein the predetermined amount of NaOH is 1 mL, 2 mL, 3 mL, 4 mL, 5 mL, or is between 1 mL and 5 mL and the concentration of NaOH is 0.1M, 0.2M, 0.3M, 0.4M, 0.5M, 0.6M, 0.7M, 0.8M, 0.9M, 1M, is between 0.1M and 2M, is between 0.1M and 1M.

53. The method of any one of claims 46-52, wherein the first predetermined period of time is 30 seconds, 1 minute, 5 minutes, 10 minutes, 15 minutes, 30 minutes, or between 30 seconds and 2 minutes, between 1-15 minutes, or between 1 and 30 minutes.

54. The method of any one of claims 46-53, wherein the second predetermined period of time is less than 30 minutes, 1-30 minutes, less than 45 minutes, between 1-45 minutes, less than 60 minutes, between 1-60 minutes, about 15 minutes, about 30 minutes, or about 1 hour.

55. The method of any one of claims 46-54, wherein the predetermined amount of Sheather's sugar solution is 1 mL, 2 mL, 3 mL, 4 mL, 5 mL, or is between 1 mL and 5 mL.

56. The method of any one of claims 46-55, wherein the second predetermined period of time is 30 seconds, 1 minute, 5 minutes, 10 minutes, 15 minutes, 30 minutes, or between 30 seconds and 2 minutes, between 1-15 minutes, or between 1 and 30 minutes.

57. The method of any one of claims 46-56, wherein aliquot of a predetermined amount is 200 μL, 210 μL, 220 μL, 230 μL 240 μL, 250 μL, 260 μL, 270 μL, 280 μL, 290 μL, 300 μL, between 200-300 μL, or between 260-300 μL.

58. The method of any one of claims 46-57, wherein the tube of predetermined size is a 1.7 mL to 2 mL tube.

59. The method of any one of claims 46-58, the predetermined amount of ferrofluid added to the tube is 10 μL 20 μL, 30 μL, 40 μL, 50 μL, 60 μL or between 10-60 μL.

60. The method of any one of claim 46-49 or 51-59, the predetermined amount of concentrated SYBR to the tube is 1 μL, 2 μL, 3 μL, 4 μL, 5 μL or between 1-5 μL.

61. The method of claims 46-60, wherein the method is performed using a microfluidic device.

62. The method of claims 46-61, wherein the method is performed using a ferrofluid-based microfluidic device.

63. The method of claims 31-62 wherein the parasite oocysts or eggs are counted using at least one of a hemocytometer and McMaster chamber.

64. The method of claim 63, where at least one of the hemocytometer and McMaster chamber are configured to cause fluorescence excitation/emission of fluorophore labeled parasite oocysts or eggs in the samples using wavelengths specific to the fluorophore used.

65. The method claims 31-64, wherein the method further comprises flowing at least one of the plurality of samples into cartridge for a predetermined period of time.

66. The method of any one of claims 31-65, wherein the method further comprises counting the oocysts and/or eggs without capture.

67. The method of any one of claims 31-66, wherein the method further comprises characterizing the counted parasite oocysts and/or eggs.

68. The method of any one of claims 31-67, wherein the method further comprises characterizing a level of sporulation of individual oocysts and/or eggs contained in at least one sample.

69. The method of any one of claims 31-68, wherein the method further comprises determining a state of oocyst and/or egg sporulation.

70. The method of any one of claims 31-69, wherein the method further comprises visualizing a level of sporulation of individual oocysts contained in at least one sample.

71. The method of claim 70, wherein sporulation is visualized by exposing the at least one sample with at least one staining agent, the at least one staining agent may comprise a DNA intercalating dye, and wherein the DNA intercalating dye can comprise SYBR.

72. The method, system or device of any one of the preceding claims, further configured to assess a size distribution of Eimeria oocysts in vaccine preparation.
